SEQUENCE LISTING (57 sequences)

SEQ ID NO: 1 - protein, 385 aa, Cannabis sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 2 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 3 - protein, 395 aa, Cannabis sativa
MGLSSVCTFSFQTNYHTLLNPHNNNPKTSLLCYRHPKTPIKYSYNNFPSKHCSTKSFHLQNKCSESLSIAKNSIRAATTNQTEPPESDNHSVATKILNFGKACWKLQRPYTIIAFTSCACGLFGKELLHNTNLISWSLMFKAFFFLVAILCIASFTTTINQIYDLHIDRINKPDLPLASGEISVNTAWIMSIIVALFGLIITIKMKGGPLYIFGYCFGIFGGIVYSVPPFRWKQNPSTAFLLNFLAHIITNFTFYYASRAALGLPFELRPSFTFLLAFMKSMGSALALIKDASDVEGDTKFGISTLASKYGSRNLTLFCSGIVLLSYVAAILAGIIWPQAFNSNVMLLSHAILAFWLILQTRDFALTNYDPEAGRRFYEFMWKLYYAEYLVYVFI

SEQ ID NO: 4 - protein, 720 aa, Cannabis sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 5 - protein, 545 aa, Cannabis sativa
MNCSAFSFWFVCKIIFFFLSFHIQISIANPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 6 - protein, 544 aa, Cannabis sativa
MKCSTFSFWFVCKIIFFFFSFNIQTSIANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 7 - protein, 3147 aa, Dictyostelium discoideum
MNKNSKIQSPNSSDVAVIGVGFRFPGNSNDPESLWNNLLDGFDAITQVPKERWATSFREMGLIKNKFGGFLKDSEWKNFDPLFFGIGPKEAPFIDPQQRLLLSIVWESLEDAYIRPDELRGSNTGVFIGVSNNDYTKLGFQDNYSISPYTMTGSNSSLNSNRISYCFDFRGPSITVDTACSSSLVSVNLGVQSIQMGECKIAICGGVNALFDPSTSVAFSKLGVLSENGRCNSFSDQASGYVRSEGAGVVVLKSLEQAKLDGDRIYGVIKGVSSNEDGASNGDKNSLTTPSCEAQSINISKAMEKASLSPSDIYYIEAHGTGTPVGDPIEVKALSKIFSNSNNNQLNNFSTDGNDNDDDDDDNTSPEPLLIGSFKSNIGHLESAAGIASLIKCCLMLKNRMLVPSINCSNLNPSIPFDQYNISVIREIRQFPTDKLVNIGINSFGFGGSNCHLIIQEYNNNFKNNSTICNNNNNNNNNIDYLIPISSKTKKSLDKYLILIKTNSNYHKDISFDDFVKFQIKSKQYNLSNRMTTIANDWNSFIKGSNEFHNLIESKDGEGGSSSSNRGIDSANQINTTTTSTINDIEPLLVFVFCGQGPQWNGMIKTLYNSENVFKNTVDHVDSILYKYFGYSILNVLSKIDDNDDSINHPIVAQPSLFLLQIGLVELFKYWGIYPSISVGHSFGEVSSYYLSGIISLETACKIVYVRSSNQNKTMGSGKMLVVSMGFKQWNDQFSAEWSDIEIACYNAPDSIVVTGNEERLKELSIKLSDESNQIFNTFLRSPCSFHSSHQEVIKGSMFEELSNLQSTGETEIPLFSTVTGRQVLSGHVTAQHIYDNVREPVLFQKTIESITSYIKSHYPSNQKVIYVEIAPHPTLFSLIKKSIPSSNKNSSSVLCPLNRKENSNNSYKKFVSQLYFNGVNVDFNFQLNSICDNVNNDHHLNNVKQNSFKETTNSLPRYQWEQDEYWSEPLISRKNRLEGPTTSLLGHRIIYSFPVFQSVLDLQSDNYKYLLDHLVNGKPVFPGAGYLDIIIEFFDYQKQQLNSSDSSNSYIINVDKIQFLNPIHLTENKLQTLQSSFEPIVTKKSAFSVNFFIKDTVEDQSKVKSMSDETWTNTCKATISLEQQQPSPSSTLTLSKKQDLQILRNRCDISKLDKFELYDKISKNLGLQYNSLFQVVDTIETGKDCSFATLSLPEDTLFTTILNPCLLDNCFHGLLTLINEKGSFVVESISSVSIYLENIGSFNQTSVGNVQFYLYTTISKATSFSSEGTCKLFTKDGSLILSIGKFIIKSTNPKSTKTNETIESPLDETFSIEWQSKDSPIPTPQQIQQQSPLNSNPSFIRSTILKDIQFEQYCSSIIHKELINHEKYKNQQSFDINSLENHLNDDQLMESLSISKEYLRFFTRIISIIKQYPKILNEKELKELKEIIELKYPSEVQLLEFEVIEKVSMIIPKLLFENDKQSSMTLFQDNLLTRFYSNSNSTRFYLERVSEMVLESIRPIVREKRVFRILEIGAGTGSLSNVVLTKLNTYLSTLNSNGGSGYNIIIEYTFTDISANFIIGEIQETMCNLYPNVTFKFSVLDLEKEIINSSDFLMGDYDIVLMAYVIHAVSNIKFSIEQLYKLLSPRGWLLCIEPKSNVVFSDLVFGCFNQWWNYYDDIRTTHCSLSESQWNQLLLNQSLNNESSSSSNCYGGFSNVSFIGGEKDVDSHSFILHCQKESISQMKLATTINNGLSSGSIVIVLNSQQLTNMKSYPKVIEYIQEATSLCKTIEIIDSKDVLNSTNSVLEKIQKSLLVFCLLGYDLLENNYQEQSFEYVKLLNLISTTASSSNDKKPPKVLLITKQSERISRSFYSRSLIGISRTSMNEYPNLSITSIDLDTNDYSLQSLLKPIFSNSKFSDNEFIFKKGLMFVSRIFKNKQLLESSNAFETDSSNLYCKASSDLSYKYAIKQSMLTENQIEIKVECVGINFKDNLFYKGLLPQEIFRMGDIYNPPYGLECSGVITRIGSNVTEYSVGQNVFGFARHSLGSHVVTNKDLVILKPDTISFSEAASIPVVYCTAWYSLFNIGQLSNEESILIHSATGGVGLASLNLLKMKNQQQQPLTNVYATVGSNEKKKFLIDNFNNLFKEDGENIFSTRDKEYSNQLESKIDVILNTLSGEFVESNFKSLRSFGRLIDLSATHVYANQQIGLGNFKFDHLYSAVDLERLIDEKPKLLQSILQRITNSIVNGSLEKIPITIFPSTETKDAIELLSKRSHIGKVVVDCTDISKCNPVGDVITNFSMRLPKPNYQLNLNSTLLITGQSGLSIPLLNWLLSKSGGNVKNVVIISKSTMKWKLQTMISHFVSGFGIHFNYVQVDISNYDALSEAIKQLPSDLPPITSVFHLAAIYNDVPMDQVTMSTVESVHNPKVLGAVNLHRISVSFGWKLNHFVLFSSITAITGYPDQSIYNSANSILDALSNFRRFMGLPSFSINLGPMKDEGKVSTNKSIKKLFKSRGLPSLSLNKLFGLLEVVINNPSNHVIPSQLICSPIDFKTYIESFSTMRPKLLHLQPTISKQQSSIINDSTKASSNISLQDKITSKVSDLLSIPISKINFDHPLKHYGLDSLLTVQFKSWIDKEFEKNLFTHIQLATISINSFLEKVNGLSTNNNNNNNSNVKSSPSIVKEEIVTLDKDQQPLLLKEHQHIIISPDIRINKPKRESLIRTPILNKFNQITESIITPSTPSLSQSDVLKTPPIKSLNNTKNSSLINTPPIQSVQQHQKQQQKVQVIQQQQQPLSRLSYKSNNNSFVLGIGISVPGEPISQQSLKDSISNDFSDKAETNEKVKRIFEQSQIKTRHLVRDYTKPENSIKFRHLETITDVNNQFKKVVPDLAQQACLRALKDWGGDKGDITHIVSVTSTGIIIPDVNFKLIDLLGLNKDVERVSLNLMGCLAGLSSLRTAASLAKASPRNRILVVCTEVCSLHFSNTDGGDQMVASSIFADGSAAYIIGCNPRIEETPLYEVMCSINRSFPNTENAMVWDLEKEGWNLGLDASIPIVIGSGIEAFVDTLLDKAKLQTSTAISAKDCEFLIHTGGKSILMNIENSLGIDPKQTKNTWDVYHAYGNMSSASVIFVMDHARKSKSLPTYSISLAFGPGLAFEGCFLKNVV

SEQ ID NO: 8 - protein, 2968 aa, Dictyostelium discoideum
MNNNKSINDLSGNSNNNIANSNINNYNNLIKKEPIAIIGIGCRFPGNVSNYSDFVNIIKNGSDCLTKIPDDRWNADIISRKQWKLNNRIGGYLKNIDQFDNQFFGISPKEAQHIDPQQRLLLHLAIETLEDGKISLDEIKGKKVGVFIGSSSGDYLRGFDSSEINQFTTPGTNSSFLSNRLSYFLDVNGPSMTVNTACSASMVAIHLGLQSLWNGESELSMVGGVNIISSPLQSLDFGKAGLLNQETDGRCYSFDPRASGYVRSEGGGILLLKPLSAALRDNDEIYSLLLNSANNSNGKTPTGITSPRSLCQEKLIQQLLRESSDQFSIDDIGYFECHGTGTQMGDLNEITAIGKSIGMLKSHDDPLIIGSVKASIGHLEGASGICGVIKSIICLKEKILPQQCKFSSYNPKIPFETLNLKVLTKTQPWNNSKRICGVNSFGVGGSNSSLFLSSFDKSTTITEPTTTTTIESLPSSSSSFDNLSVSSSISTNNDNDKVSNIVNNRYGSSIDVITLSVTSPDKEDLKIRANDVLESIKTLDDNFKIRDISNLTNIRTSHFSNRVAIIGDSIDSIKLNLQSFIKGENNNNKSIILPLINNGNNNNNNNNNSSGSSSSSSNNNNICFIFSGQGQQWNKMIFDLYENNKTFKNEMNNFSKQFEMISGWSIIDKLYNSGGGGNEELINETWLAQPSIVAVQYSLIKLFSKDIGIEGSIVLGHSLGELMAAYYCGIINDFNDLLKLLYIRSTLQNKTNGSGRMHVCLSSKAEIEQLISQLGFNGRIVICGNNTMKSCTISGDNESMNQFTKLISSQQYGSVVHKEVRTNSAFHSHQMDIIKDEFFKLFNQYFPTNQISTNQIYDGKSFYSTCYGKYLTPIECKQLLSSPNYWWKNIRESVLFKESIEQILQNHQQSLTFIEITCHPILNYFLSQLLKSSSKSNTLLLSTLSKNSNSIDQLLILCSKLYVNNLSSIKWNWFYDKQQQQQSESLVSSNFKLPGRRWKLEKYWIENCQRQMDRIKPPMFISLDRKLFSVTPSFEVRLNQDRFQYLNDHQIQDIPLVPFSFYIELVYASIFNSISTTTTNTTASTMFEIENFTIDSSIIIDQKKSTLIGINFNSDLTKFEIGSINSIGSGSSSNNNFIENKWKIHSNGIIKYGTNYLKSNSKSNSFNESTTTTTTTTTTTKCFKSFNSNEFYNEIIKYNYNYKSTFQCVKEFKQFDKQGTFYYSEIQFKKNDKQVIDQLLSKQLPSDFRCIHPCLLDAVLQSAIIPATNKTNCSWIPIKIGKLSVNIPSNSYFNFKDQLLYCLIKPSTSTSTSPSTYFSSDIQVFDKKNNNLICELTNLEFKGINSSSSSSSSSSTINSNVEANYESKIEETNHDEDEDEELPLVSEYVWCKEELINQSIKFTDNYQTVIFCSTNLNGNDLLDSIITSALENGHDENKIFIVSPPPVESDQYNNRIIINYTNNESDFDALFAIINSTTSISGKSGLFSTRFIILPNFNSITFSSGNSTPLITNVNGNGNGKSCGGGGGSTNNTISNSSSSISSIDNGNNEDEEMVLKSFNDSNLSLFHLQKSIIKNNIKGRLFLITNGGQSISSSTPTSTYNDQSYVNLSQYQLIGQIRVFSNEYPIMECSMIDIQDSTRIDLITDQLNSTKLSKLEIAFRDNIGYSYKLLKPSIFDNSSLPSSSSEIETTATTKDEEKNNSINYNNNYYRVELSDNGIISDLKIKQFRQMKCGVGQVLVRVEMCTLNFRDILKSLGRDYDPIHLNSMGDEFSGKVIEIGEGVNNLSVGQYVFGINMSKSMGSFVCCNSDLVFPIPIPTPSSSSSSNENIDDQEIISKLLNQYCTIPIVFLTSWYSIVIQGRLKKGEKILIHSGCGGVGLATIQISMMIGAEIHVTVGSNEKKQYLIKEFGIDEKRIYSSRSLQFYNDLMVNTDGQGVDMVLNSLSGEYLEKSIQCLSQYGRFIEIGKKDIYSNSSIHLEPFKNNLSFFAVDIAQMTENRRDYLREIMIDQLLPCFKNGSLKPLNQHCFNSPCDLVKAIRFMSSGNHIGKILINWSNLNNDKQFINHHSVVHLPIQSFSNRSTYIFTGFGGLTQTLLKYFSTESDLTNVIIVSKNGLDDNSGSGSGNNEKLKLINQLKESGLNVLVEKCDLSSIKQVYKLFNKIFDNDASGSDSGDFSDIKGIFHFASLINDKRILKHNLESFNYVYNSKATSAWNLHQVSLKYNLNLDHFQTIGSVITILGNIGQSNYTCANRFVEGLTHLRIGMGLKSSCIHLASIPDVGMASNDNVLNDLNSMGFVPFQSLNEMNLGFKKLLSSPNPIVVLGEINVDRFIEATPNFRAKDNFIITSLFNRIDPLLLVNESQDFIINNNINNNGGGGDGSFDDLNQLEDEGQQGFGNGDGYVDDNIDSVSMLSGTSSIFDNDFYTKSIRGMLCDILELKDKDLNNTVSFSDYGLDSLLSSELSNTIQKNFSILIPSLTLVDNSTINSTVELIKNKLKNSTTSSISSSVSKKVSFKKNTQPLIIPTTAPISIIKTQSYIKSEIIESLPISSSTTIKPLVFDNLVYSSSSSNNSNSKNELTSPPPSAKRESVLPIISEDNNSDNDSSMATVIYEISPIAAPYHRYQTDVLKEITQLTPHKEFIDNIYKKSKIRSRYCFNDFSEKSMADINKLDAGERVALFREQTYQTVINAGKTVIERAGIDPMLISHVVGVTSTGIMAPSFDVVLIDKLGLSINTSRTMINFMGCGAAVNSMRAATAYAKLKPGTFVLVVAVEASATCMKFNFDSRSDLLSQAIFTDGCVATLVTCQPKSSLVGKLEIIDDLSYLMPDSRDALNLFIGPTGIDLDLRPELPIAINRHINSAITSWLKKNSLQKSDIEFFATHPGGAKIISAVHEGLGLSPEDLSDSYEVMKRYGNMIGVSTYYVLRRILDKNQTLLQEGSLGYNYGMAMAFSPGASIEAILFKLIK

SEQ ID NO: 9 - protein, 307 aa, Streptomyces Sp. Strain Cl190 [sequence omitted]

SEQ ID NO: 10 - protein, 398 aa, Cannabis sativa
MGLSLVCTFSFQTNYHTLLNPHNKNPKNSLLSYQHPKTPIIKSSYDNFPSKYCLTKNFHLLGLNSHNRISSQSRSIRAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 11 - protein, 413 aa, Humulus lupulus
MELSSVSSFSLGTNPFISIPHNNNNLKVSSYCCKSKSRVINSTNSKHCSPNNNSNNNTSNKTTHLLGLYGQSRCLLKPLSFISCNDQRGNSIRASAQIEDRPPESGNLSALTNVKDFVSVCWEYVRPYTAKGVIICSSCLFGRELLENPNLFSWPLIFRALLGMLAILGSCFYTAGINQIFDMDIDRINKPDLPLVSGRISVESAWLLTLSPAIIGFILILKLNSGPLLTSLYCLAILSGTIYSVPPFRWKKNPITAFLCILMIHAGLNFSVYYASRAALGLAFAWSPSFSFITAFITFMTLTLASSKDLSDINGDRKFGVETFATKLGAKNITLLGTGLLLLNYVAAISTAIIWPKAFKSNIMLLSHAILAFSLIFQARELDRTNYTPEACKSFYEFIWILFSAEYVVYLFI

SEQ ID NO: 12 - protein, 139 aa, Phaeodactylum tricornutum
MRLTQGCFSFLPDLTDQQIEKQIAYCITKGWAMNVEWTDDPHPRNSYWELWGLPLFDVKDPASVMFELREARKSCAAGYIRINAFNAAYGTESCVMSFIVNRPSNEPGFYLERQELEGRRIAYTTKSYSVQANPEGGRY

SEQ ID NO: 13 - protein, 251 aa, Aequorea victoria
MRKGEELFTGVVPILVELDGDVNGHKFSVSGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSYQSALSKDPNEKRDHMVLLEFVTAAGITHGMDELYKRPAANDENYAASV

SEQ ID NO: 14 - protein, 236 aa, Discosoma sp.
MRSSKNVIKEFMRFKVRMEGTVNGHEFEIEGEGEGRPYEGHNTVKLKVTKGGPLPFAWDILSPQFQYGSKVYVKHPADIPDYKKLSFPEGFKWERVMNFEDGGVVTVTQDPSLQDGCFIYKVKFIGVNFPSDGPVMQKKTMGWEASTERLYPRDGVLKGEIHKALKLKDGGHYLVEFKTIYMAKKPVQLPGYYYVDSKLDITSHNKDYTIVEQYERTEGRHHLFLKAELGSNVGER

SEQ ID NO: 15 - DNA, 482 bp, Artificial Sequence
  FBAC2-1 intron
gtacgtacgcgtaacatattgtagccaatttggtgtcgacggcatggtctcgcagggaacgatagaaaaacgttgacacctagaaacgggggctctggcacggcagctctccacggattctctcgcagtattacacgggctatgcagtggacagggataccaaacgtatgttggtgtcttaatgtaaactttgcccgtaaattccgtccatatcgatcgaatccttaccgtcaagggagacctccagttcccatggtcgaggggctttcgtggaccatcccgccgcaagatccatccgctggtgtgacgtcgagcagcgccgaacggtgtcagtgacgtggcatccctcccccatccacagcaaacacgagtagttttggtcgcgtttataccgcgctccaaaccccagaattggccgtcgcggttttccgtaccgttggtctcacactggtcccgcgttttttctttgattccacaatcag

SEQ ID NO: 16 - DNA, 136 bp, Artificial Sequence
  TUFA-1 intron
gtaagactctgcaggctccacgtgaacgagtacctcgaacggtatggtaccgtcacaatacacggtttctgcccttgttagctcacacgtttgctgtcctttctactcgttcttccctgttgttgatccttgttag

SEQ ID NO: 17 - DNA, 92 bp, Artificial Sequence
  EIF6-1 intron
gtacgttttgttgtggtctattgacaactgtagagtgcgtgaagcatttacatcttgaaatggtacatctgacgctttttgtcatcttgaag

SEQ ID NO: 18 - DNA, 196 bp, Artificial Sequence
  RPS4-1 intron
gtaagaatactcattcttcgtcaatgagattgttgagtctctgataggaaccgaaaatgtaggaaggaagcgctggcaactttctgatgaaagatgtttctgatgaaagacgtttgccgttgacaaacatccgtcccacgaaagtagtgtcgggaaacgttggctcactcggttgattctttttctcctttaatag

SEQ ID NO: 19 - DNA, 145 bp, Artificial Sequence
  RbcS2-2 intron
gtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcag

SEQ ID NO: 20 - DNA, 329 bp, Artificial Sequence
  RbcS2-2 intron
gtgagcttgcggggttgcgagcaacactccagcaacgaacagtgcccaagtcaggaatctgcagtcagcctgggctttcggcggctttttcttgggcaaacagcttgcactcatgccagcgcggcttgtccagcctcacttgagctttccagctgctaccagccgggctatacgacagcgacagagccatagcgtggaatcacttatttgggttgccgaagtagcggtcggagcgtgagttcttggtcaagccgccccttatccggttcctgtccgtgtctttgtccctcgttcacccttcgcggcacccttcatccccttgcttgcag

SEQ ID NO: 21 - DNA, 605 bp, Artificial Sequence
  pEF-1a
cgaaacgaatagaagctccccgaggtcgggtgttgtttgggaggttcatggtggtttcggtgtcgcttgctcgctcgctcgttcgcagtgacagacagttcgtgagacacgagaaccgttggcgtccgagttcgggtgccgcatttcgtcgtctccacgattcaattcttgcccatcagacgagtcccgaattccgtgactctggatgcgatttactttctaactgtaagcgaaactcaacgattccgtacgttgttttctattttacagtgagtcttcgataccaccgtacaaccatcgttcgtgtaccgtctggtagtcccacgtgtcgacaacgtgtggctctggaccgatgagttgtttgccgttcggaaacgagcagtaccaaggaattcacagaaacacagcccatgtaacacaacgaccgcgaatcgtttcggtgctctcgcttcgcgtacgggcgggcggtcctcccgagcagcgagaggagtccgcagcgtcatagttgcaatccgggcccccctcgcgttgttcactctctcgtctagtagagaaacttccatcggatcgtatcataatattgtatcgtataatatcacgtaatc

SEQ ID NO: 22 - DNA, 600 bp, Artificial Sequence
  p40SRPS8
ccctgcgatagaccttttccaaactcacgcagtccaagaaaacaaaggggtgagaagtatacgcacctttcggtttcggcataattcttaaactcttgtggtcactttcttgtgaagaagctaggggcactcgttttccctcagagcctgcaaacacaaaattcctgcagtcaattgtcccaacactcggcaaaccgtatgcgcaagcaacgatgcgcagaaggccgtggatggatggcgactcgcgatatggcttcttgggtcgccagtgtggtacgtccggcgtatgtcaatacgcgaattcggacgactggcatctctaggaggaggattccttcttttatgacatgtttattttatatacattgatgctttccgacagtcggaagtaataaatgaatttatttcaagactacctatactcctttgacttgttcgactaatcttaccgcttactaaaatctcgaaatcacgcttgacctctcgcacgcaaatttttgctgctggacgctacgcactcggcccaattcttctcggtcctcgtcgtcgcaattgtcgttgcgttgatcttgcaccgaaggaatcagagaatagaatacc

SEQ ID NO: 23 - DNA, 603 bp, Artificial Sequence
  pH4-1B
cgtcggtctctttcccgggaaacgggtacactcctccgcgccaacaacatattactactactaccaagaacgtccacggccttgtcgtgcgttacgctctcccaacgcgtgcggggtaaattacgtctcggtttgctaagtagcgcacagctaaatagatgaccgttattgtatttaagatcattcaatattgattgcattgtactttgcgtcaaactgaaattccctcgtactaacggttaacccgtcaaccctaagcgttcgcccaaagtagtcaaccgggacacgcgaaccgacattgggcagatctttcacagacagaaaaccatttccaatccaaataagcatgactattacacacccattcgtagcgcgaggacaaactgatagctccaacaaaatgcgccaacatcgtacattgtaagaagcttacggaacactatgtatgtagaaccatacgaacagcaactagtactggccatcgagcagcggtgactcccggctttcgtagcgctgtgaaggttacactctcacaattcgctctcggctacaaccgacaaaagtcttactcacagtcaataccgaaaacaaacaacagccaac

SEQ ID NO: 24 - DNA, 600 bp, Artificial Sequence
  py-Tubulin
ttcgttgatatttttattcaaatgtatcgggaggagtagaggttgattaactgtaaacaatttcctatttactgttaaggaccagctgctgcagtaggtatggcctatccactaaacgcactcacggaacgcctcgcgaaatttacccacggccaacttacattaccgccttttgtgaattggaaacgccgcatgattctcaaatgcgcagaatttcaaacggtagcttgcggtggagactcgctcattgacagtgaaactaccttgtgtcctcggattttcagatatacctatacagttcatggcaaaatttcgttcatgaacgcacgtgatccattgctcgcgattcccgtttttgattgtgaacgcgggattacatgcgtgcggtgacggtagtccagacacagatatttgcaataccgggcccttttcactacagaccctgtaggggtatgttgacgagaatgaactcgcagactgccaaaatcgctttggctgatcccaagttttggcactccatcgtaatttgtcatattccatacggtagcttcgactgaatccagacaaacaatttagtccagctgcgcttctacttgcaat

SEQ ID NO: 25 - DNA, 504 bp, Artificial Sequence
  pRBCMT
acacggaggatctatctacagcagcgatgagggcgcccgagaaagaaagaacgattgccgtactattctctttgacctttgggcgctcgctcgtatctttgaagcgactgttggggtctcagggtccaaaaaacagaaactggattgacagtgtgtctggaccttgtcgaaccttacagttacattacagttaattgtcactgtaaatagtctatcgctggattacgtcatcgcgtgactgggtgggaatccttcttgttgacagtgaatctacggtatactattccttgggcgcttgtacttgtgtctcgagattgccgacagtgacgtcaattcggcacccacaccttccacccgccgaaccaaaatcaacaacacgaagcacacgaccgaccgactgtacacgtgaaggagcaaaccatcgaacgaaaggagccttccacggacacaacccgaaagctcgacacccttcacccacgcaaagtatctcttcgtgatcctacc

SEQ ID NO: 26 - DNA, 250 bp, Artificial Sequence
  pFcpB
gaaacataccttcagcgtcgtcttcactgtcacagtcaactgacagtaatctttggcccgtagaggttcgaaattcaatctattaaatacagcaggataagacacaagagcgacatcctgacatcaactccgtgaacagcaaatcctggttgaacacgtatccttttgggggcctccagctacgacgctcgccccagctggggcttccttactatacacagcgcatatttcgcggttgccagaagtcaag

SEQ ID NO: 27 - DNA, 599 bp, Artificial Sequence
  pFcpC
gagcacaagaggtgacaaaagccaccggctggatcgcacttctcggaatttcccccctactatcaaacaaattcgaattgccaaaggtgaagggactaactgtaaatcctgatcaatcaaggtctcaatcaagtacaatgggctacaatgatatttagatgggaacacaatgaaacaaattgaaacttctactgacaggagcgcaattgacttgtgtagcttttcatgagcacttgattgctaccaattgtgaacgggatggggaaagactcgaaaaggtgcatgcttccgataatctactatattttctagaatcaaataatatttaaatgaatgaggtcctcagcgtacgttaagcctacttatttagaacgagaagtcagaccgaggggtactaaaattctaagggttgagaggtatcttgattccgggtctatggaagcccatccttgttgaagcttgaacacgatccttgtgaaaggccgacgttgcgcgaaaaaacagcctgccgatttctttccttctttctcgtctcaacctatatactttcataatctctgttagagtttaccaacaacacatatatacatttcgacaaa

SEQ ID NO: 28 - DNA, 593 bp, Artificial Sequence
  pFcpD
actagcttgattgggatatctcgctcatgtttgtcgcgtgctatgtctttttaggtactttgaacctacgttcgtacttgtataatatgatcatcgtattatcgtttttcatccgtccagcgcaaaatgcattagcagctagtcctagcgtgcggagctacctggacaggtgcatgacggatgcgtgtccttcagtgactttctaattaacagtaacttctttacttatgtttcagtttgtaagaagcgggattcgctcgtcggttgacatctgattggactgcgtcggcacatgaaaactacattgtgaaatctgctaaaactccgggtatctctgacacaaaacgattcggcttcgcaatttcaacattacggtcaaggctaacgtatctttctcggtcaacttcagattatgccgattaaattgtcgtagctttcaaggcgttttgagtactgcggcagttgttgaacctgcaaggagaagatctcgacaacagaataaagcgaaaaatgggtctcatgcactaacactcaggcctccctcataatctctgtttgagtttaccaacaacacatatatacatttcgacaaa

SEQ ID NO: 29 - DNA, 499 bp, Artificial Sequence
  tEF-1a
atgcgggagtggaccgcgacgatccgtccggaaaacaatactaggtgctatcacaggggcgcgttttggagagacgttctgcggaaacacgaatttagaatacgtaactaacatataaactggatagccctcgcatcggaacttagaatgttcgcctcaatttttagtttagcgtggagcagagatacctttccatttggcaaaatctacctttcgtgagggacatcttgagaaataagcggacttgtagactaggaccgtggtaacctcctctcaatctaccaatgttgtctgatttccgagccgcgcggctgaaaatcgtctagcacttggatgcgagagcaaatgtcaagtcctgctctgtcctgttggacgctttcctctcaccgcgagagggctttcactcgcgaaacacgtatttcatattcaaactctatgaagtttaaagtagatgtatctacaaacggtcctaagtttgggtaagaattttcgactgcat

SEQ ID NO: 30 - DNA, 499 bp, Artificial Sequence
  t40SRPS8
agataagaatatctcattgtgaacatctatgatttaccaattttattctttgtttacagttagacgccagtaattgtgctgtttctctcaagtctgtgtcaatacaaactacgaaacttggcaatttttctcttgaatatgagcacgagattgaaacgcacaaaggaaattagtttccatcctttgacaaagtttgttgctgtttagagaacagatgtcaaaattaacgtgccatggaattgaaccatggtgcgctatccccaaatcacgtcgtttgacctcgtcacatttaaggtatatcaagcatattcacttatatcttgacatcctccctgcttgatattcctttgcccttgagccatcttccccacacgctggagatgacccctgcccctcctttctttatccacccgagtaaggtgtcgacaagtcactttgcccctgagccatcctccccactcaggagatgacccctacccctttcttctttatccacccc

SEQ ID NO: 31 - DNA, 500 bp, Artificial Sequence
  tH4-1B
gcttgcgcttgatcctcgactttgttgctcgtctttaaaaccttggaacgattatatacaatcaccaactcaaaaaccggattttctaaaatccacccaaacaaccaaagaaaatacttctcattgcatttatgaatcacagcagacctgcgtccttttaaaacttagatcctgttttcttataaaaaacagatcaaattttctgggagttcattgactctgccagtcagaatcaatcctgcagtaattctttatttacaggtgaaagtaaaagagaatcccaattttttgcttgtacattaaggtcctccttacattacagctaatttcaaaataagatgaagttggattcgtgtcctttcatggtgatgtgatattctgcactataccaaacaccgtgaaatgtcagctagagcttgtcatgaggcagttgctgccaatcactacatatagatccttcacggagaaaagttggcttcattctctgttgctaatcggct

SEQ ID NO: 32 - DNA, 500 bp, Artificial Sequence
  ty-Tubulin
agcaaactcattatgatgcatgggagtgcgaccgagtttcgaacgatgctaacgaacattattagtggaaggcagtcattgttgtatgcgtcaaagtatataatcagacggacagtagttcattttaaacttttgttcggaaagcgttgatcattcatcggggaatcgcgctaacgagcagtaattggagttgtaactgcaggctcaacgcgtttctggctcccgtgggttaccataatgctaactatcattctttatttgcagtatcaccagtccaagaattattcgtggtcatttctgatgctactgtggagaagtgagagtaatttcgccgactttgaagtgaagacgcgtctccgaacgtagattgttggtattgtaccattgaaggagagtttttgaactcaggttgttgatcgtaatgtccgcgacatcctggcgcactacacgccaatgaccataacatgtcttggtcgccctcctcgtaagtcatcgccatt

SEQ ID NO: 33 - DNA, 501 bp, Artificial Sequence
  tRBCMT
aaatacaaattcatgtacctaaacgatagtatggatgatgggagtaattgcactataattgtagaaccttgtaagaggaaaaaatgatcttactgtgttatttcctcttgaaagaatctatggataaaataagagaggacgctaggtggtaacattccggcaaaacactggcgcctaaatttttgccggaatcgtcaattgcaacggttgtaccggtgctttgttttagtgtttcgccttgctacccttcaggaaccggtaacgaacacctccgccaccccggacccgctctgttttagtttgaatggacttgcgcaaagaatccttatcattcgcctgtgaccgggttccttccttggccaaatttgccagaaaggtttcccgatcaatgctaggggtgcttccgcctccacttgccgtgggagtgttcccaccgctacttgccgttggagtgctatcactatcggagccttggatcgtgttgggcgtaccggtagtgcc

SEQ ID NO: 34 - DNA, 502 bp, Artificial Sequence
  tFcpB
tttacttgctgggtaggccgtttctggaataacatattagattctaactggttcgaagcattgcgttgctgtaacattcccgttcacaaaaatacagaacagtctagaagttcgcgacgacataatttttctctttaggaggccggggttgtaattgttctagggctgttccaatagagaagataagatgatcaaacataccagccgcgcttgattggacggagtacgtttgcatcagctatttttcaaaagcgctgcacgacgcacactctatgaacacttcaagactctcaacgcaagtgacaaccatcctctccaaaaggctatctttcggggcacctgtaatataaaaaagcatggcagtgcattccatgcaaaaaatgtctaatctggttgggttttaaagtccgtatcgagcacagaggtgacaaaagccaccggctggatcgcacttctcggaatttcccccctactatcaaacaaattcgaattgccaaaggtg

SEQ ID NO: 35 - DNA, 492 bp, Artificial Sequence
  tFcpC
ttttgttacattgacttcaaggagtcgaggaatcgatactgccgtcgtttccaggatccgaggtttctatagactctctatagactctgttaacctaatagaatcagacatacctctcctgctattttgtttttatgaatttggcttttgcctctctagtcagatttgaatgttattttccgccaggtgtgttagtcgggctctcgtttgagttacaagagggattgagtggcgaggattcactctaatgtaaatatgactgtgaacaaaactttaaaattactacgcatcttctttgactgtcagatattcgtcggtgacagcagtcaatgcctgcaaattgtcctcctgggtcgcaatttggttttggattgacctggtatgcattatgaagaaaaaaattcgttattagccaactgcctagcgtgcacattgcatggttagacctccttgacgactgtgagcctacatccttctgcaacaagctgcaat

SEQ ID NO: 36 - DNA, 336 bp, Artificial Sequence
  tFcpD
ttttgttacatttactgacttcaaggagtcgaggaatcgatactgccgtcgtttccaggatccgaggtttcataaactctgttaacgttatagaaacagacttacctctcctacgccattcacgtaatattcgcaatatgctattcttcctctgaagaccaggtttatgtgctgcctgaaactatttcaataagtcagctgcacttgcacagggtttcacaaggaaagcgtgtctttttttccaacgtaggcgtcgctttcgtctgactcttactcttacattcacagccaatacttacaattagtaaaaaacctgtgctcgagagtgaaaacgtc

SEQ ID NO: 37 - protein, 6 aa, Artificial Sequence
  6His
HHHHHH

SEQ ID NO: 38 - protein, 10 aa, Artificial Sequence
  Myc
EQKLISEEDL

SEQ ID NO: 39 - protein, 8 aa, Artificial Sequence
  FLAG
DYKDDDDK

SEQ ID NO: 40 - protein, 14 aa, Artificial Sequence
  V5
GKPIPNPLLGLDST

SEQ ID NO: 41 - protein, 9 aa, Artificial Sequence
  HA
YPYDVPDYA

SEQ ID NO: 42 - protein, 12 aa, Artificial Sequence
  HSV
QPELAPEDPEDC

SEQ ID NO: 43 - protein, 24 aa, Artificial Sequence
  FMDV2A
APVKQTLNFDLLKLAGDVESNPGP

SEQ ID NO: 44 - protein, 40 aa, Artificial Sequence
  extFMDV2a
LLAIHPTEARHKQKIVAPVKQTLNFDLLKLAGDVESNPGP

SEQ ID NO: 45 - protein, 21 aa, Artificial Sequence
  T2A
GSGEGRGSLLTCGDVEENPGP

SEQ ID NO: 46 - protein, 15 aa, Artificial Sequence
  3(GGGGS)
GGGGSGGGGSGGGGS

SEQ ID NO: 47 - protein, 12 aa, Artificial Sequence
  FPL1
GGGGSSGGGGGS

SEQ ID NO: 48 - protein, 15 aa, Artificial Sequence
  FPL1
GGGGSGGGGSGGGGS

SEQ ID NO: 49 - DNA, 1524 bp, Artificial Sequence
  Ptref1
atgaaccacctccgtgccgaaggacccgcctccgtcctcgccattggaaccgccaaccccgaaaacattctcctccaggacgaattcccggactactacttccgcgtcaccaagtcggaacacatgacccagctcaaggaaaagttccgtaagatctgtgacaagtcgatgattcgtaagcgcaactgcttcctcaacgaagaacacttgaagcagaacccccgtctcgtcgaacacgaaatgcagaccctcgacgcccgtcaggacatgctcgtcgtcgaagtccccaagctcggaaaggatgcctgcgccaaggccatcaaggaatggggtcagcccaagtccaagatcacccacctcattttcacctccgcctccaccaccgacatgcccggagctgactaccactgcgccaagttgctcggcctctccccctccgtcaagcgcgtcatgatgtaccagcttggctgctacggcggaggtaccgtcttgcgtattgccaaggacatcgccgaaaacaacaagggagcccgtgtcttggccgtctgctgtgacattatggcctgcctcttccgtggcccctccgaatccgacctcgaactcttggtcggtcaggccatctttggcgatggagccgccgccgtcattgtcggtgccgaacccgacgaatccgtcggagaacgtcccatttttgaacttgtctccaccggacagaccattctccccaactccgaaggaaccattggcggccacattcgtgaagccggactcatctttgacctccacaaggacgtcccgatgctcatctccaacaacatcgaaaagtgcctcattgaagccttcactccgatcggaatttccgactggaactccatcttctggattacccaccccggcggaaaggccattttggacaaggtcgaagaaaagctccacctcaagtccgataagtttgtcgactcccgtcacgtcctctccgaacacggtaacatgtcctcctccaccgtcctctttgtcatggacgaactccgtaagcgttccttggaagaaggcaagtcgaccaccggcgacggcttcgaatggggagtcttgtttggcttcggtcccggattgaccgtcgaacgtgtcgtcgtccgttccgttcccatcaagtacggatccggagaaggacgcggttcgttgctcacctgcggagacgtcgaagaaaaccccggacccatggccgtcaagcacctcattgtcttgaagttcaaggatgaaatcaccgaagctcagaaggaagaattcttcaagacctacgtcaacctcgtcaacatcattcccgccatgaaggacgtctactggggaaaggacgtcacccagaagaacaaggaagaaggatacacccacattgtcgaagtcacctttgaatccgtcgaaaccatccaggactacatcatccaccccgcccacgtcggattcggtgacgtctaccgctccttctgggaaaagctcttgattttcgactacaccccccgtaaataa

SEQ ID NO: 50 - DNA, 1506 bp, Artificial Sequence
  Ptref2
atgaaccacctccgtgccgaaggacccgcctccgtcttggccattggtaccgctaaccccgaaaacattttgctccaggacgaatttcccgactactactttcgtgtcactaagtccgaacacatgacccagttgaaggaaaagtttcgtaagatttgcgacaagtccatgatccgtaagcgtaactgctttctcaacgaagaacacctcaagcagaacccgcgtctcgtcgaacacgaaatgcagaccctcgacgcccgccaggacatgctcgtcgtcgaagtccccaagctcggaaaggacgcctgcgctaaggccattaaggaatggggacagcccaagtccaagatcacccacttgatcttcacctccgcctccaccaccgacatgcccggtgccgactaccactgtgccaagctccttggactctccccgtccgtcaagcgtgtcatgatgtaccagctcggttgttacggaggaggcaccgttttgcgtattgccaaggacattgccgaaaacaacaagggagcccgcgtcttggccgtctgctgcgacattatggcctgcctcttccgtggtccgtccgaatccgacctcgaactcctcgtcggtcaggccattttcggagacggagccgccgccgtgattgtcggagccgaacccgatgaatccgtcggcgaacgtcccattttcgaactcgtctccaccggacagacgatcttgcccaactccgaaggcaccattggaggacacattcgtgaagccggcctcattttcgacctccacaaggatgtccccatgctcatttcgaacaatattgaaaagtgcttgatcgaagccttcacccccattggaatttcggactggaactccattttctggattacccaccccggaggcaaggccattttggacaaggtcgaagaaaagctccacttgaagtccgacaagttcgtcgactcccgccacgtcttgtcggaacacggtaacatgtcgtcctccaccgtcctcttcgtgatggatgaattgcgtaagcgttccctcgaagaaggtaaatccaccaccggagacggttttgaatggggtgtcctctttggtttcggacccggattgaccgtcgaacgtgtcgtcgtccgttccgtccccattaagtacggaggtggaggttccggaggaggtggatccggaggtggaggttcgatggccgtcaagcacctcattgtcctcaagttcaaggatgaaattaccgaagcccagaaggaagaattctttaagacctacgtcaacttggtcaacattattccggccatgaaggacgtctactggggaaaggatgtcacccagaagaacaaggaagaaggatacacccacattgtcgaagtcacctttgaatccgtcgaaaccattcaggactacattattcaccccgcccacgtcggatttggagacgtctaccgttccttttgggaaaagctcttgatttttgactacaccccccgtaaataa

SEQ ID NO: 51 - DNA, 1572 bp, Artificial Sequence
  Ptref3
atgaaccacctccgtgccgaaggtcccgcctccgtcctcgccattggaaccgccaaccccgaaaacattctcctccaagacgaattccctgactactacttccgcgtgactaagagtgagcacatgacgcaattgaaagaaaagttccgtaagatctgtgataagtccatgatccgtaagcgtaactgtttcctgaatgaggaacaccttaagcagaacccccgcttggtcgaacatgaaatgcagaccttggatgcccgccaggacatgctcgtcgttgaagtccctaagttgggtaaagacgcttgcgccaaggccattaaggaatggggacagcccaagagtaagatcacccatttgattttcacatccgcctctacgaccgatatgcctggagccgattaccactgcgccaagcttctgggactgtccccctccgttaagcgcgtcatgatgtaccagcttggatgttacggaggtggcaccgtcctccgtattgccaaggacattgccgaaaacaacaagggtgcccgtgtcctcgccgtctgctgcgacatcatggcctgcctcttccgcggaccctccgaatccgacttggaactcctcgtgggacaggccatttttggtgacggcgccgccgccgtcattgtcggagctgaacccgacgaatccgtcggagaaaggcccatttttgaattggtctccaccggacagaccattctcccgaactccgaaggaaccatcggcggacacattcgtgaagccggtctcatctttgacttgcacaaggacgttcccatgctcatttccaacaacattgaaaagtgcctcattgaagccttcacccccattggaatctccgactggaatagtatcttttggattacccaccccggaggaaaggccatcttggataaggtggaagaaaagctccacctcaagtccgacaagttcgtcgactcccgtcacgtcttgtccgaacacggaaacatgtcgtcctccaccgtcctctttgtcatggatgaattgcgtaagcgctcgctcgaagaaggaaagtccaccaccggcgatggtttcgaatggggcgttctcttcggatttggacccggtctcaccgtcgaacgtgtcgtcgtccgttctgtcccgattaagtaccaccatcaccaccaccacggatcgggcgaaggacgtggttccctccttacgtgcggagacgtcgaagaaaaccccggtcccatggccgtcaagcacttgatcgttctcaagttcaaggacgaaatcaccgaagcccagaaggaagaattcttcaagacctacgtcaacctcgtcaacatcattcccgccatgaaggatgtttactggggaaaggacgtcacccagaagaacaaggaagaaggttacacccacatcgtcgaagtcacctttgaatccgttgaaaccatccaggactacattatccacccggcccacgtcggcttcggagacgtctaccgttccttttgggaaaagttgttgattttcgactacaccccccgtaaggaacagaagttgatttccgaagaagacctctaa

SEQ ID NO: 52 - DNA, 2292 bp, Artificial Sequence
  Ptref7
atgcgtaagggtgaagaactcttcaccggagtcgtccccatcttggtggaattggacggagacgtcaacggccataagttctcggtttccggtgaaggtgaaggtgacgccactaacggaaagctcaccctcaagttcatttgcaccaccggcaagctcccggtcccctggcccaccctcgtcaccaccttgacttacggtgtccagtgcttcgcccgttaccccgaccacatgaagcagcacgacttctttaagtccgccatgcccgaaggatacgtccaggaacgcaccatttccttcaaggacgacggaacctacaagacccgtgccgaagtcaagtttgaaggagataccctcgtcaaccgcattgaactcaagggcattgatttcaaagaagatggcaacattctcggacacaagctcgaatacaacttcaactcccacaacgtctacatcaccgccgataagcaaaagaacggtattaaggccaacttcaagattcgccacaacgtcgaagacggttccgtccagttggccgaccactaccagcagaacaccccgattggtgacggtccggtcctcctccccgacaaccactacctctcgtaccagtccgccctctccaaggatccgaacgagaagcgtgaccacatggtcctcttggaattcgtcaccgccgccggaatcacccacggcatggacgaactctacaagcgtcccgccgccaacgacgagaactacgccgcctccgtcggtatgaaccacttgcgcgccgaaggaccggcttccgtcttggccattggtaccgccaacccggaaaacatcctcctccaggacgaattccccgactactacttccgtgtcaccaagtccgaacacatgacccagttgaaggaaaaattccgtaagatttgtgataagtccatgattcgtaagcgtaactgcttcttgaacgaagaacacctcaagcaaaacccccgtctcgtcgaacacgaaatgcaaaccctcgacgcccgtcaggacatgctcgttgtcgaagtcccgaagcttggcaaggacgcctgcgccaaggccattaaggaatggggccagcccaagagcaagattacccacttgattttcacctccgcctccaccaccgatatgcccggcgctgactaccactgcgctaagctcctcggtctctccccctccgtcaagcgtgtcatgatgtaccagctcggatgctacggaggtggaaccgtcctccgcattgccaaggacattgctgaaaacaacaagggcgcccgtgtcctcgccgtctgctgtgacattatggcctgcttgtttcgtggtccctccgaatccgatttggaactcctcgtcggccaggccatttttggtgacggtgccgccgccgtcatcgtcggcgccgaaccggacgaatccgtcggtgaacgtccgatttttgaactcgtctccaccggtcaaaccattctccccaactccgaaggaaccatcggaggacacatccgtgaagccggactcatttttgacctccacaaggatgtccccatgctcatctccaacaacatcgaaaagtgcctcatcgaagccttcacccccattggcatctccgactggaactccattttctggattacccaccccggtggaaaggccattctcgacaaggtcgaagaaaagctccacttgaagtccgataagttcgtcgactcccgccacgtcctctccgaacacggaaacatgtcctcctccaccgtcctcttcgtcatggacgaactccgtaagcgttccctcgaagaaggaaagtccacgacgggtgacggattcgagtggggagtcctcttcggcttcggtcccggactcaccgtcgaacgtgtcgtcgtccgttccgtgcccatcaagtacggaggaggaggatccggtggtggcggatccggaggtggtggatcgatggccgtcaagcacctcattgtcttgaagtttaaggacgaaatcaccgaagcccagaaggaagaattcttcaagacttacgtcaacttggtcaacatcatccccgccatgaaggacgtctactggggaaaggacgtcacccaaaagaacaaggaagaaggatacacccacattgtcgaagtcacctttgaatccgtcgaaaccattcaggactacattattcaccccgcccacgtcggtttcggtgacgtctaccgttccttctgggaaaagctcttgatttttgactacaccccccgtaaggaacagaagttgatttccgaagaagacttgtaa

SEQ ID NO: 53 - DNA, 452 bp, Artificial Sequence
  pFcpA
ctgcaggacgcaatggaggattatcaccgcaaaaatgaacttcgaaaaaaactttcgagcgaccatggaaaaggaggatcagattcagattacaacagtggattgctctggtagcaaatatcttctgctagattggctcatggtcggttttggacgttcgaagctcaccgtcaaaagaaacaaaagagaagaatgacgtcttcgtgacgtagaatctacgactgtactcggatctgggaaatgaattgactcacggtcttcttcgagtcctgttacaggcccttggtccgaacccccacacgatttttgcaccaaagatttgcttcaatttgctggatgttttgactgcaagatcagctggcctagcaagagtgctcgtgttgcttcgtcgggaatccctacgaatttcagttctgcacaaatttgtctgccgtttcgagatatcattcaag

SEQ ID NO: 54 - DNA, 299 bp, Artificial Sequence
  tFcpA
ccgacgccgaccaacaccgccggtccgacgcggcccgacgggtccgaggcctcggagatctgggcccatgcggccgcaacaactacctcgactttggctgggacactttcagtgaggacaagaagcttcagaagcgtgctatcgaactcaaccagggacgtgcggcacaaatgggcatccttgctctcatggtgcacgaacagttgggagtctctatccttccttaaaaatttaattttcattagttgcagtcactccgctttggtttcacagtcaggaataacactagctcgtcttca

SEQ ID NO: 55 - DNA, 461 bp, Artificial Sequence
  pHSP70A-RbcS2
tcgctgaggcttgacatgattggtgcgtatgtttgtatgaagctacaggactgatttggcgggctatgagggcgggggaagctctggaagggccgcgatggggcgcgcggcgtccagaaggcgccatacggcccgctggcggcacccatccggtataaaagcccgcgaccccgaacggtgacctccactttcagcgacaaacgagcacttatacatacgcgactattctgccgctatacataaccactcagctagcttaagatcccatcaagcttgcatgccgggcgcgccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttcttt

SEQ ID NO: 56 - DNA, 239 bp, Artificial Sequence
  tRbcS2
ggatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttgaagc

SEQ ID NO: 57 - DNA, 1533 bp, Artificial Sequence
  G1C1 construct
atgaaccacctgcgggcggagggccccgcctccgtgctggcgatcggcactgccaaccccgagaacatcctgctgcaggacgagtttcctgactactactttcgcgtgacgaagtcggagcacatgacccagctcaaggagaagtttcgcaagatttgcgacaagagcatgatccgcaagcgcaactgcttcctgaacgaggagcacctgaagcagaacccccgcctggtggagcacgagatgcagaccctcgacgctcggcaggacatgctcgtggtggaggtgcccaagctgggtaaggacgcctgcgcgaaggccatcaaggagtggggccaacccaagtcgaagatcacccacctgatcttcacgagcgcctcgacgaccgacatgcccggcgccgattaccactgcgccaagctgctgggcctgtcgccgtcggtgaagcgcgtgatgatgtaccagctgggttgctacggcggcggcactgtcctgcggatcgctaaggacatcgcggagaacaacaagggcgcgcgcgtcctggcggtctgctgcgacatcatggcctgcctgttccggggcccctccgagagcgacctggagctgctggtgggccaggcgattttcggcgacggcgcggccgcggtgatcgtcggcgccgagccggacgagagcgtgggggagcgccccatcttcgagctggtgtccaccggccagaccatcctccctaacagcgagggcaccatcggcggccacatccgggaggccggcctgattttcgacctgcacaaggacgtccccatgctgatctcgaacaacatcgagaagtgcctgatcgaggctttcactcccatcggcatctccgactggaactccatcttctggatcacgcacccgggcggcaaggccattctggacaaggtggaggagaagctgcacctgaagagcgacaagtttgtggacagccgccacgtcctgagcgagcacgggaacatgtccagctcgacggtgctgtttgtcatggacgagctgcgcaagcggagcctggaggagggcaagtcgactaccggtgacggcttcgagtggggggtgctgttcggcttcggccccggcctgacggtcgagcgggtggtggtccgcagcgtgccgatcaagtacgcgcccgtgaagcagaccctgaacttcgacctgctgaagctggcgggcgatgtggagtccaaccccggccccatggccgtgaagcacctcatcgtgctgaagttcaaggacgagatcacggaggcccagaaggaggagtttttcaagacttacgtgaacctggtcaacatcatccctgcgatgaaggacgtctactggggcaaggacgtcacccagaagaacaaggaggagggctacacccatattgtggaggtgacctttgagtcggtcgagaccatccaggactacatcatccaccctgcccacgtgggctttggcgacgtgtaccgctcgttttgggagaagctgctgattttcgactacaccccccgcaagtaa

(19) United States
(12) Patent Application Publication
(10) Pub. No.: US 2023/0014531 A1
(43) Pub. Date: Jan. 19, 2023

(54) Engineered Microorganism for the Production of Cannabinoids

(71) Applicant: Algae-C Inc., Caledon (CA)

(72) Inventors: Isabel Desgagné-Penix, Trois-Rivières (CA); Fatima Awwad, Trois-Rivières (CA); Manel Ghribi, Trois-Rivières (CA)

(21) Appl. No.: 17/772,710

(22) PCT Filed: Oct. 29, 2020
(86) PCT No.: PCT/CA2020/051452

§ 371 Date: Apr. 28, 2022

Related U.S. Application Data
(60) Provisional application No. 62/927,321, filed on Oct. 29, 2019.

(51) Int. Cl. C12P 17/06; C12N 9/10; C12N 9/88; C12N 1/12; C12N 9/02

(52) U.S. Cl. CPC C12P 17/06; C12N 9/1029; C12N 9/88; C12N 9/1085; C12N 1/12; C12N 9/0004; C12Y 404/01026; C12R 2001/89

(57) ABSTRACT

A genetically engineered microorganism for the production of a cannabinoid is described. The genetically engineered microorganism comprises at least one nucleic acid molecule encoding at least one cannabinoid biosynthetic pathway enzyme. The disclosure also relates to methods for producing a cannabinoid using a genetically engineered microorganism.

### CROSS-REFERENCE TO RELATED APPLICATIONS

This application claims the benefit of and priority from United States Provisional Patent Application No. 62/927,321, filed Oct. 29, 2019.

### FIELD

The present disclosure relates to genetically engineered microorganisms for production of cannabinoids and cell cultures comprising thereof. The genetically engineered microorganisms comprise nucleic acid molecules having nucleic acid sequences encoding cannabinoid biosynthetic pathway enzymes for producing cannabinoid biosynthetic pathway products.

### BACKGROUND

The commercialization of valuable plant natural products (PNPs) is often limited by the availability of PNP producing-plants, by the low accumulation of PNPs in planta and/or the time-consuming and often inefficient extraction methods not always economically viable. Thus, commercialization of PNPs of commercial interest is often challenging. The recent progress in genetic engineering and synthetic biology makes it possible to produce heterologous PNPs in microbes such as bacteria, yeasts and microalgae. For example, engineered microorganisms have been reported to produce the antimalarial drug artemisinin and of the opiate (morphine, codeine) painkiller precursor reticuline (Keasling 2012; Fossati et al 2014; DeLoache et al 2015). However, the latest metabolic reactions to yield the valuable end-products such as codeine and morphine in genetically modified yeast-producing reticuline have yet to be successfully achieved. In some cases, bacterial or yeast platforms do not support the assembly of complex PNP pathways. In comparison, microalgal cells have been suggested to possess advantages over other microorganisms, including the likelihood to perform similar post-translational modifications of proteins as plant and recombinant protein expression through the nuclear, mitochondrial or chloroplastic genomes (Singh et al 2009).

Δ9-tetrahydrocanannabinol and other cannabinoids (CBs) are polyketides responsible for the psychoactive and medicinal properties of Cannabis sativa. More than 110 CBs have been identified so far and are all derived from fatty acid and terpenoid precursors (ElSohly and Slade 2005). The first metabolite intermediate in the CB biosynthetic pathway in Cannabis sativa is olivetolic acid that forms the polyketide skeleton of cannabinoids. A type III polyketide synthase (PKS; also known as tetraketide synthase (TKS) or olivetol synthase) enzyme condenses hexanoyl-CoA with three malonyl-CoA in a multi-step reaction to form trioxododecanoyl-CoA. From there, olivetolic acid cyclase (OAC) (OAC; also known as 3,5,7-trioxododecanoyl-CoA CoA-lyase) catalyzes an intramolecular aldol condensation to yield OA. In subsequent steps, CB diversification is generated by the sequential action of “decorating” enzymes on the OA backbone. The gene sequence for PKS and OAC have been identified and characterized in vitro (Lussier 2012; Gagne et al 2012; Marks et al 2009; Stout et al 2012; Taura et al 2009).

### SUMMARY

The present disclosure describes an engineered microorganism such as a microalga or a cyanobacterium for production of a plant natural product such as a cannabinoid.

In an embodiment of the genetically engineered microorganism as described herein, the genetically engineered microorganism does not comprise an exogenous nucleic acid molecule encoding aromatic prenyltransferase.

The present disclosure also provides a cell culture comprising the genetically engineered microorganism as described herein, and a medium that is substantially free of a sugar.

The present disclosure also provides a method for producing a cannabinoid in a genetically engineered microorganism, comprising introducing into the microorganism at least one nucleic acid molecule encoding tetraketide synthase and olivetolic acid cyclase, wherein the microorganism is a microalga or a cyanobacterium.

The present disclosure also provides a method for producing a cannabinoid in a genetically engineered microorganism, comprising introducing into the microorganism at least one nucleic acid molecule encoding Steely1, Steely 2, or a variant thereof, wherein the microorganism is a microalga or a cyanobacterium.

The present disclosure also provides a method for producing a cannabinoid in a wild type microorganism, comprising culturing the microorganism in a medium comprising a 2,4-dihydroxy-6-alkylbenzoic acid or a 2,4-dihydroxy-6-alkylbenzoate, wherein the microorganism is a microalga or a cyanobacterium.

Other features and advantages of the present disclosure will become apparent from the following detailed description. It should be understood, however, that the detailed description and the specific Examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The disclosure will now be described in relation to the drawings in which:

FIG. 1 shows an exemplary cannabinoid biosynthetic pathway based on enzymes from Cannabis sativa.

FIG. 2 shows a part of the cannabinoid biosynthetic pathway from Cannabis sativa ending in the production of olivetolic acid.

FIG. 3 shows the concentration of the mobile phase as a function of time in the HPLC procedure for detecting cannabinoids in transformed cells.

FIG. 4 shows the standard curves for (A) THC, (B) CBD, and (C) CBN determined by diluting 0, 5, 10, 25, 50, 75, and 100 ppm concentrations of the cannabinoid in solvent made of wild type P.tricornutum extract. For each curve, the red line represents the standard curve calculated from the height of the peak and the blue line represents the standard curve calculated from the area of the peak at each concentration.

FIG. 5 shows the HPLC chromatograms (280 nm) for the detection of cannabinoids in P.tricornutum transformed with constructs (A) Ptref1, (B) Ptref2, (C) Ptref3, (D) Ptref7. (E) HPLC chromatogram for a wild type P.tricornutum control is shown. The retention time (Rt) for authentic standard of cannabinoids are THC, 17.8-18.4 min; CBD 15.09-15.4 min; and CBN: 17-17.5 min.

FIG. 6 shows the UPLC chromatograms (220 nm) of (A) C.reinhardtii transformed with construct G1C1 and (B) control sample with cannabinoid standards.

FIG. 7 shows the UPLC chromatograms (220 nm) for the detection of cannabinoids in P.tricornutum transformed with constructs (A) Ptref1, (B) Ptref2, and (C) wild-type P.tricornutum control.

### DETAILED DESCRIPTION

It has been surprisingly discovered by the present inventors that microalgae, for example Phaeodactylum tricornutum and Chlamydomonas reinhardtii, transformed with an exogenous nucleic acid molecule that encodes tetraketide synthase and olivetolic acid cyclase produced cannabinoids in the absence of any other exogenous cannabinoid biosynthetic pathway enzymes.

Without wishing to be bound by theory, it is expected that the genome of microalgae contains genes that encode enzymes with similar activity to enzymes found in Cannabis sativa (e.g. aromatic prenyltransferase (APT), tetrahydrocannabinolic acid synthase (THCAS), and/or cannabidiolic acid synthase (CBDAS)) that allow for the production of cannabinoids in the presence of a precursor such as, for example, olivetol or olivetolic acid. For example, searching the genome of P.tricornutum strain CCAP 1055/1 (NCBI BLAST) identifies a predicted protein (NCBI Reference Sequence XP 002182033.1) with 36% shared identity over 81% query cover to a region of APT containing active sites (amino acids 108-383, APT). This predicted protein shares sequence identity with a homogentisate solanesyltransferase enzyme that is capable of prenyltransfer, shares sequence identity with hydroxybenzoate polyprenyltransferase, and contains conserved magnesium binding sites similar to APT from Cannabis sativa. Other potential candidates for APT activity in P.tricornutum include geranyl geranyl transferase, and a predicted protein (NCBI Reference Sequence: XP 002180392.1). Furthermore, searching the genome of P.tricornutum for enzymes that produce H₂O₂ identifies violaxanthin deepoxidase-like protein and spermine oxidase that may have activity similar to CBDAS.

Accordingly, the present disclosure provides a genetically engineered microorganism that is capable of producing a cannabinoid, wherein the genetically engineered microorganism is a photosynthetic microalga or a cyanobacterium, and wherein the genetically engineered microorganism does not comprise an exogenous nucleic acid molecule encoding aromatic prenyltransferase.

The present disclosure further provides a cell culture comprising a genetically engineered microorganism for production of a cannabinoid, and a medium that is substantially free of a sugar, wherein the genetically engineered microorganism is a photosynthetic microalga or a cyanobacterium, and wherein the genetically engineered microorganism does not comprise an exogenous nucleic acid molecule encoding aromatic prenyltransferase.

The present disclosure further provides a method for producing a cannabinoid in a genetically engineered microorganism, comprising introducing into the microorganism at least one nucleic acid molecule encoding tetraketide synthase and olivetolic acid cyclase, wherein the microorganism is a microalga or a cyanobacterium.

The present disclosure further provides a method for producing a cannabinoid in a genetically engineered microorganism, comprising introducing into the microorganism at least one nucleic acid molecule encoding Steely1, Steely 2, or a variant thereof, wherein the microorganism is a microalga or a cyanobacterium.

The present disclosure further provides a method for producing a cannabinoid in a wild type microorganism, comprising culturing the microorganism in a medium comprising a 2,4-dihydroxy-6-alkylbenzoic acid or a 2,4-dihydroxy-6-alkylbenzoate, wherein the microorganism is a microalga or a cyanobacterium.

Unless otherwise indicated, the definitions and embodiments described in this and other sections are intended to be applicable to all embodiments and aspects of the present disclosure herein described for which they are suitable as would be understood by a person skilled in the art.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

As used herein, the singular forms “a”, “an” and “the” include plural references unless the content clearly dictates otherwise. In embodiments comprising an “additional” or “second” component, the second component as used herein is different from the other components or first component. A “third” component is different from the other, first, and second components, and further enumerated or “additional” components are similarly different.

In the absence of any indication to the contrary, reference made to a “%” content throughout this specification is to be taken as meaning % w/v (weight/volume).

As used here, the term “sequence identity” refers to the percentage of sequence identity between two nucleic acid (polynucleotide) or two amino acid (polypeptide) sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions multiplied by 100%). In one embodiment, the two sequences are the same length. The determination of percent identity between two sequences can also be accomplished using a mathematical algorithm. One non-limiting example of a mathematical algorithm utilized for the comparison of two sequences is the algorithm of Karlin and Altschul (1990), modified as in Karlin and Altschul (1993). Such an algorithm is incorporated into the NBLAST and XBLAST programs of Altschul et al (1990). BLAST nucleotide searches can be performed with the NBLAST nucleotide program parameters set, e.g., for score=100, wordlength=12 to obtain nucleotide sequences homologous to a nucleic acid molecules of the present disclosure. BLAST protein searches can be performed with the XBLAST program parameters set, e.g., to score=50, wordlength=3 to obtain amino acid sequences homologous to a protein molecule of the present disclosure. To obtain gapped alignments for comparison purposes, Gapped BLAST can be utilized as described in Altschul et al. (1997). Alternatively, PSI-BLAST can be used to perform an iterated search which detects distant relationships between molecules (Altschul et al., 1997). When utilizing BLAST, Gapped BLAST, and PSI-Blast programs, the default parameters of the respective programs (e.g., of XBLAST and NBLAST) can be used (see, e.g., the NCBI website). Another non-limiting example of a mathematical algorithm utilized for the comparison of sequences is the algorithm of Myers and Miller (1988). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted. In a specific embodiment, the nucleic acids are optimized for codon usage in a specific microalgal or cyanobacterial species. In particular, the nucleic acid sequence encoding the cannabinoid biosynthetic pathway enzyme incorporates codon-optimized codons for GC-rich microalgae, such as Chlamydomonas reinhardtii, Chlorella vulgaris, Chlorella sorokiniana, Chlorella protothecoides, Tetraselmis chui, Nannochloropsis oculate, Scenedesmus obliquus, Acutodesmus dimorphus, Dunaliella tertiolecta, and Heamatococus plucialis; diatoms, such as Phaeodactylum tricornutum and Thalassiosira pseudonana; or cyanobacteria such as Arthrospira platensis, Arthrospira maxima, Synechococcus elongatus, and Aphanizomenon flos-aquae.

The sequences of the present disclosure may be at least 80% identical to the sequences described herein; in another example, the sequences may be at least 80%, 85%, 90%, 95%, 96%, 97%, 98%, 99%, or 100% identical at the nucleic acid or amino acid level to sequences described herein. Importantly, the proteins encoded by the variant sequences retain the activity and specificity of the proteins encoded by the reference sequences. Accordingly, the present disclosure also provides a nucleic acid molecule comprising nucleic acid sequence encoding a cannabinoid biosynthetic pathway enzyme with at least 80%, 85%, 90%, 95%, 96%, 97%, 98%, 99% or 100% sequence identity to a sequence selected from SEQ ID NO:49-52. Also provided is an amino acid sequence of a cannabinoid biosynthetic pathway enzyme with at least 90%, 91%, 92%, 93%, 94%, 95%, 96%, 97%, 98%, 99%, or 100% sequence identity to a sequence selected from SEQ ID NO:1-11.

Nucleic acid and amino acid sequences described herein are set out in Table 1.

TABLE 1
Sequences

SEQ ID NO: 1 amino acid sequence of tetraketide synthase from Cannabis sativa
SEQ ID NO: 2 amino acid sequence of olivetolic acid cyclase from Cannabis sativa
SEQ ID NO: 3 amino acid sequence of aromatic prenyltransferase (CsPT1) from
Cannabis sativa
SEQ ID NO: 4 amino acid sequence of hexanoyl-CoA synthetase from Cannabis sativa
SEQ ID NO: 5 amino acid sequence of tetrahydrocannabinolic acid synthase from
Cannabis sativa
SEQ ID NO: 6 amino acid sequence of cannabidiolic acid synthase from Cannabis
sativa
SEQ ID NO: 7 amino acid sequence of Steely1 from Dictyostelium discoideum
SEQ ID NO: 8 amino acid sequence of Steely2 from Dictyostelium discoideum
SEQ ID NO: 9 amino acid sequence of Orf2 from Streptomyces Sp. Strain CI190
SEQ ID NO: 10 amino acid sequence of CsPT4 from Cannabis sativa
SEQ ID NO: 11 amino acid sequence of HIPT1 from Humulus lupulus
SEQ ID NO: 12 amino acid sequence of Rubisco small subunit
SEQ ID NO: 13 amino acid sequence of yellow fluorescent reporter (YFP)
SEQ ID NO: 14 amino acid sequence of red fluorescent protein (RFP)
SEQ ID NO: 15 nucleic acid sequence of FBAC2-1 Intron
SEQ ID NO: 16 nucleic acid sequence of TUFA-1 Intron
SEQ ID NO: 17 nucleic acid sequence of EIF6-1 Intron
SEQ ID NO: 18 nucleic acid sequence of RPS4-1 Intron
SEQ ID NO: 19 nucleic acid sequence of RbcS2-1 intron
SEQ ID NO: 20 nucleic acid sequence of RbcS2-2 intron
SEQ ID NO: 21 nucleic acid sequence of Elongation Factor-1 alpha Promoter pEF-1α
SEQ ID NO: 22 nucleic acid sequence of 40SRPS8 Promoter p40SRPS8
SEQ ID NO: 23 nucleic acid sequence of Histone H4 Promoter pH4-1B
SEQ ID NO: 24 nucleic acid sequence of Tubulin gamma chain Promoter pγ-Tubulin
SEQ ID NO: 25 nucleic acid sequence of Ribulose-1,5-bisphosphate
carboxylase/oxygenase small subunit N-methyltransferase I Promoter pRBCMT
SEQ ID NO: 26 nucleic acid sequence of Fucoxanthin-chlorophyll a/c binding protein B
Promoter pFcpB
SEQ ID NO: 27 nucleic acid sequence of Fucoxanthin-chlorophyll a/c binding protein C
Promoter pFcpC
SEQ ID NO: 28 nucleic acid sequence of Fucoxanthin-chlorophyll a/c binding protein D
Promoter pFcpD
SEQ ID NO: 29 nucleic acid sequence of Elongation Factor-1 alpha Terminator tEF-1α
SEQ ID NO: 30 nucleic acid sequence of 40SRPS8 Terminator t40SRPS8
SEQ ID NO: 31 nucleic acid sequence of Histone H4 Terminator tH4-1B
SEQ ID NO: 32 nucleic acid sequence of Tubulin gamma chain Terminator tγ-Tubulin
SEQ ID NO: 33 nucleic acid sequence of Ribulose-1,5-bisphosphate
carboxylase/oxygenase small subunit N-methyltransferase I Terminator tRBCMT
SEQ ID NO: 34 nucleic acid sequence of carboxylase/oxygenase small subunit N-
methyltransferase I Terminator tFcpB
SEQ ID NO: 35 nucleic acid sequence of Fucoxanthin-chlorophyll a/c binding protein B
Terminator tFcpC
SEQ ID NO: 36 nucleic acid sequence of Fucoxanthin-chlorophyll a/c binding protein C
Terminator tFcpD
SEQ ID NO: 37 amino acid sequence of 6His tag
SEQ ID NO: 38 amino acid sequence of Myc tag
SEQ ID NO: 39 amino acid sequence of FLAG tag
SEQ ID NO: 40 amino acid sequence of V5 tag
SEQ ID NO: 41 amino acid sequence of HA tag
SEQ ID NO: 42 amino acid sequence of HSV tag
SEQ ID NO: 43 amino acid sequence of FMDV2a linker peptide
SEQ ID NO: 44 amino acid sequence of extFMDV2a linker peptide
SEQ ID NO: 45 amino acid sequence of T2A linker peptide
SEQ ID NO: 46 amino acid sequence of 3(GGGGS) linker peptide
SEQ ID NO: 47 amino acid sequence of FPL1 linker peptide
SEQ ID NO: 48 amino acid sequence of FPL1 linker peptide
SEQ ID NO: 49 nucleic acid sequence of construct Ptref1
SEQ ID NO: 50 nucleic acid sequence of construct Ptref2
SEQ ID NO: 51 nucleic acid sequence of construct Ptref3
SEQ ID NO: 52 nucleic acid sequence of construct Ptref7
SEQ ID NO: 53 nucleic acid sequence of Fucoxanthin-chlorophyll a/c binding protein A
Promoter pFcpA
SEQ ID NO: 54 nucleic acid sequence of Fucoxanthin-chlorophyll a/c binding protein A
Terminator tFcpA
SEQ ID NO: 55 nucleic acid sequence of HSP70A-RbcS2 Hybrid Promoter
SEQ ID NO: 56 nucleic acid sequence of RbcS2 Terminator
SEQ ID NO: 57 nucleic acid sequence of construct G1C1

As used herein, the term “genetically engineered” and its derivatives refer to a microorganism whose genetic material has been altered using molecular biology techniques such as but not limited to molecular cloning, recombinant DNA methods, transformation and gene transfer. The genetically engineered microorganism includes a living modified microorganism, genetically modified microorganism or a transgenic microorganism. Genetic alteration includes addition, deletion, modification and/or mutation of genetic material. Such genetic engineering as described herein in the present disclosure increases production of plant natural products such as cannabinoids relative to the corresponding wild-type microorganism. The term “cannabinoid” is generally understood to include any chemical compound that acts upon a cannabinoid receptor. Examples of cannabinoids include cannabidiol (CBD), cannabinol (CBN), cannabigerol (CBG), cannabichromene (CBC), tetrahydrocannabivarin (THCV), cannabichromanon (CBCN), cannabielsoin (CBE), cannbifuran (CBF), tetrahydrocannabinol (THC), cannabinodiol (CBDL), cannabicyclol (CBL), cannabitriol (CBT), cannabivarin (CBV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinodiol (CBND), cannabinol propyl variant (CBNV), cannabitriol (CBO), cannabigerolic acid (CBGA), tetrahydrocannabinolic acid (THCA), cannabichromenic acid (CBCA), tetrahydrocannabivarinic acid (THCVA), cannabigerovarinic acid (CBGVA), cannabidivarinic acid (CBDVA), cannabichromevarinic acid (CBCVA), and derivatives thereof. Further examples of cannabinoids are discussed in PCT Patent Application Pub. No. WO2017/190249 and US Patent Application Pub. No. US2014/0271940.

A cannabinoid may be in an acid form or a non-acid form, the latter also being referred to as the decarboxylated form since the non-acid form can be generated by decarboxylating the acid form. Within the context of the present disclosure, where reference is made to a particular cannabinoid, the cannabinoid can be in its acid or non-acid form, or be a mixture of both acid and non-acid forms.

A cannabinoid biosynthetic pathway product is a product associated with the production of cannabinoids. Examples of cannabinoid biosynthetic pathway products include, but are not limited to hexanoyl-CoA, butyryl-CoA, trioxododecanoyl-CoA, trioxodecanoyl-CoA, olivetolic acid, olivetol, divarinolic acid, and divarinol. In an embodiment, the cannabinoid biosynthetic pathway product is at least one, two, three, four, five, six, seven, or eight of hexanoyl-CoA, butyryl-CoA, trioxododecanoyl-CoA, trioxodecanoyl-CoA, olivetolic acid, olivetol, divarinolic acid, and divarinol.

In one embodiment, the genetically engineered microorganism has increased production of at least one, two, three, four, five, six, seven, or eight cannabinoid biosynthetic pathway products relative to the corresponding wild-type microorganism. In another embodiment, the cannabinoid biosynthetic pathway product is at least one, two, three, four, five, six, seven, or eight of hexanoyl-CoA, butyryl-CoA, trioxododecanoyl-CoA, trioxodecanoyl-CoA, olivetolic acid, olivetol, divarinolic acid, and divarinol. For example, the genetically engineered microorganism may have increased production of olivetolic acid, or olivetolic acid and cannabigerolic acid, relative to the corresponding wild-type microorganism. In another example, the genetically engineered microorganism may have increased production of olivetol, or olivetol and cannabigerol, relative to the corresponding wild-type microorganism.

The term “nucleic acid molecule” or its derivatives, as used herein, is intended to include unmodified DNA or RNA or modified DNA or RNA. For example, it is useful for the nucleic acid molecules of the disclosure to be composed of single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically double-stranded or a mixture of single- and double-stranded regions. In addition, it is useful for the nucleic acid molecules to be composed of triple-stranded regions comprising RNA or DNA or both RNA and DNA. The nucleic acid molecules of the disclosure may also contain one or more modified bases or DNA or RNA backbones modified for stability or for other reasons. “Modified” bases include, for example, tritiated bases and unusual bases such as inosine. A variety of modifications can be made to DNA and RNA; thus “nucleic acid molecule” embraces chemically, enzymatically, or metabolically modified forms. The term “polynucleotide” shall have a corresponding meaning. In some embodiments, the genetically engineered microorganism comprises at least one nucleic acid molecule described herein.

As used herein, the term “exogenous” refers to an element that has been introduced into a cell. An exogenous element can include a protein or a nucleic acid. An exogenous nucleic acid is a nucleic acid that has been introduced into a cell, such as by a method of transformation. An exogenous nucleic acid may code for the expression of an RNA and/or a protein. An exogenous nucleic acid may have been derived from the same species (homologous) or from a different species (heterologous). An exogenous nucleic acid may comprise a homologous sequence that is altered such that it is introduced into the cell in a form that is not normally found in the cell in nature. For example, an exogenous nucleic acid that is homologous may contain mutations, being operably linked to a different control region, or being integrated into a different region of the genome, relative to the endogenous version of the nucleic acid. An exogenous nucleic acid may be incorporated into the chromosomes of the transformed cell in one or more copies, into the plastid or mitochondrial DNA of the transformed cell, or be maintained as a separate nucleic acid outside of the transformed cell genome.

The term “nucleic acid sequence” as used herein refers to a sequence of nucleoside or nucleotide monomers consisting of naturally occurring bases, sugars and intersugar (backbone) linkages and includes cDNA. The term also includes modified or substituted sequences comprising non-naturally occurring monomers or portions thereof. The nucleic acid sequences of the present application may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The nucleic acid can be either double stranded or single stranded, and represents the sense or antisense strand. Further, the term “nucleic acid” includes the complementary nucleic acid sequences.

Cannabinoids produced by a genetically engineered microorganism provided herein can be the result of increasing activity of one or more enzymes associated with cannabinoid biosynthetic pathway. Increase of activity of an enzyme in a microorganism can include, for example, the introduction of a nucleic acid molecule comprising a nucleic acid sequence encoding the enzyme. In an embodiment, introduction of a nucleic acid molecule comprising a nucleic acid sequence encoding an enzyme can be accomplished by transformation. Examples of cannabinoid biosynthetic pathway enzymes include, but are not limited to hexanoyl-CoA synthetase, type III polyketide synthase (e.g., tetraketide synthase, Steely 1 and Steely 2), olivetolic acid cyclase, geranyl pyrophosphate synthase, aromatic prenyltransferase (APT), geranyl pyrophosphate:olivetolic acid geranyltransferasecannabichromene synthase, tetrahydrocannabinolic acid synthase (THCAS), and cannabidiolic acid synthase (CBDAS).

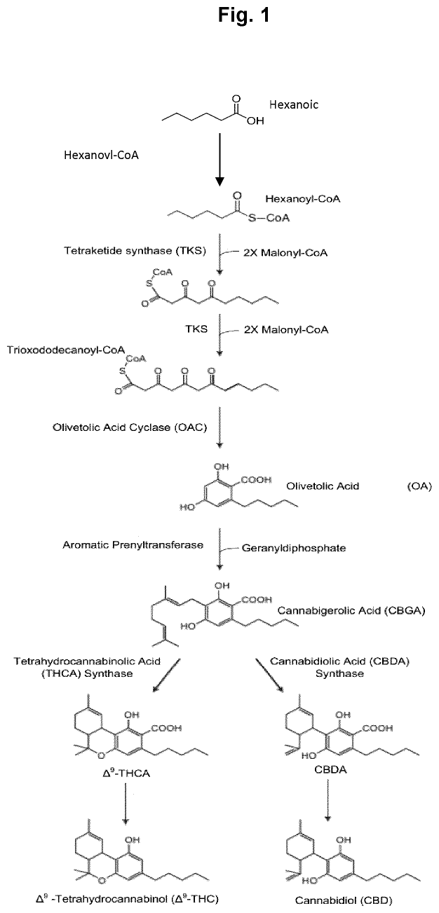

FIG. 1 shows an exemplary cannabinoid biosynthetic pathway based on enzymes from Cannabis sativa: Tetraketide synthase (TKS) condenses hexanoyl-CoA and malonyl-CoA to form the intermediate trioxododecanoyl-CoA; Olivetolic acid cyclase (OAC) catalyzes an intramolecular aldol condensation to yield olivetolic acid (OA); aromatic prenyltransferase transfers a geranyldiphosphate (GPP) onto OA to produce cannabigerolic acid (CBGA); tetrahydrocannabinolic acid synthase or cannabidiolic acid synthase catalyze the oxidative cyclization of CBGA into tetrahydrocannabinolic acid (THCA) or cannabidiolic acid (CBDA), respectively. Decarboxylation of THCA or CBDA to remove the carboxyl group will produce decarboxylated cannabinoids tetrahydrocannabinol (THC) or cannabidiol (CBD), respectively.

In addition to the exemplary cannabinoid biosynthetic pathway from Cannabis sativa shown in FIG. 1, alternative biosynthetic intermediates can be used in a cannabinoid biosynthetic pathway in a genetically engineered microorganism. For example, olivetol is an intermediate that lacks the carboxyl group of olivetolic acid. Use of olivetol instead of olivetolic acid in a cannabinoid biosynthetic pathway will produce cannabinoids that similarly lack a carboxyl group such as cannabigerol (CBG), tetrahydrocannabinol (THC), or cannabidiol (CBD). In another example, tetraketide synthase (TKS) condenses butyryl-CoA and malonyl-CoA to form the intermediate trioxodecanoyl-CoA, and olivetolic acid cyclase (OAC) catalyzes an intramolecular aldol condensation of trioxodecanoyl-CoA to yield divarinolic acid. Divarinolic acid is an intermediate containing an n-propyl group in place of the n-pentyl group found in olivetolic acid. Use of divarinolic acid instead of olivetolic acid in a cannabinoid biosynthetic pathway will produce cannabinoids that similarly contain an n-propyl group such as cannabigerovarinic acid (CBGVA), tetrahydrocannabivarinic acid (THCVA), cannabidivarinic acid (CBDVA), or cannabichromevarinic acid (CBCVA). In another example, divarinol is an intermediate that lacks the carboxyl group of divarinolic acid, and contains an n-propyl group in place of the n-pentyl group found in olivetol. Use of divarinol instead of divarinolic acid in a cannabinoid biosynthetic pathway will produce cannabinoids that similarly contain an n-propyl group and lack a carboxyl group such as cannabigerovarin (CBGV), tetrahydrocannabivarin (THCV), cannabidivarinic acid (CBDV), or cannabichromevarinic acid (CBCV).

In addition to the exemplary cannabinoid biosynthetic pathway from Cannabis sativa shown in FIG. 1, alternative enzymes can be used in a cannabinoid biosynthetic pathway in a genetically engineered microorganism. For example, in addition to the enzymes found in Cannabis sativa, alternative enzymes of a cannabinoid biosynthetic pathway may be found in other plants (e.g., Humulus lupulus), in bacteria (e.g., Streptomyces), or in protists (e.g., Dictyostelium discoideum). Enzymes that differ in structure, but perform the same function, may be used interchangeably in a cannabinoid biosynthetic pathway in a genetically engineered microorganism. For example, the aromatic prenyltransferases CsPT1 (SEQ ID NO:3) and CsPT4 (SEQ ID NO:10) from Cannabis sativa, HIPT1 from Humulus lupulus (SEQ ID NO:11), and Orf2 (SEQ ID NO:9) from Streptomyces Sp. Strain C1190 are all aromatic prenyltransferases that catalyze the synthesis of CBGA from GPP and OA. In a further example, the Steely1 (SEQ ID NO:7) or Steely2 (SEQ ID NO:8) polyketide synthase from Dictyostelium discoideum, or a variant thereof, can be used to condense malonyl-CoA into olivetol, and may be used in place of TKS to produce olivetol in the absence of OAC.

In addition to the wild-type enzymes found in organisms discussed herein, modified variants of these enzymes can be used in a cannabinoid biosynthetic pathway in a genetically engineered microorganism. Variants of enzymes for use in a cannabinoid biosynthetic pathway can be generated by altering the nucleic acid sequence encoding said enzyme to, for example, increase/decrease the activity of a domain, add/remove a domain, add/remove a signaling sequences, or to otherwise alter the activity or specificity of the enzyme. For example, the sequence of Steely1 can be modified to reduce the activity of a methyltransferase domain in order to produce non-methylated cannabinoids. By way of example, this can be done by mutating amino acids G1516D+G1518A or G1516R relative to SEQ ID NO:7 as disclosed in WO/2018/148849, herein incorporated by reference. In a further example, the sequences of tetrahydrocannabinolic acid synthase or cannabidiolic acid synthase can be modified to remove an N-terminal secretion peptide. By way of example, this can be done by removing amino acids 1-28 of SEQ ID NO:5 or 6 to produce a truncated enzyme as disclosed in WO/2018/200888, herein incorporated by reference.

A acyl-CoA synthetase is an acyl-activating enzyme that ligates CoA and a straight-chain alkanoic acid or alkanoate containing 2 to 6 carbon atoms to produce alkanoyl-CoA, wherein the alkanoyl-CoA is a thioester of coenzyme A containing an alkanoyl group of 2 to 6 carbon atoms. In one embodiment, the acyl-CoA synthetase is hexanoyl-CoA synthetase, which ligates CoA and hexanoic acid or hexanoate to produce hexanoyl-CoA. A hexanoyl-CoA synthetase may have the amino acid sequence of SEQ ID NO: 4 or an amino acid sequence with at least 90% identity to SEQ ID NO: 4. In another embodiment, an acyl-CoA synthetase ligates CoA and butyric acid or butyrate to produce butyryl-CoA.

A type III polyketide synthase is an enzyme that produces polyketides by catalyzing the condensation reaction of acetyl units to thioester-linked starter molecules. A type III polyketide synthase may have the amino acid sequence of SEQ ID NO: 1, 7 or 8 or an amino acid sequence with at least 90% identity to SEQ ID NO: 1, 7 or 8. In an embodiment, a type III polyketide synthase condenses an alkanoyl-CoA with three malonyl-CoA in a multi-step reaction to form a 3,5,7-trioxoalkanoyl-CoA, wherein the 3,5,7-trioxoalkanoyl-CoA contains 8 to 12 carbon atoms. In another embodiment, the type III polyketide synthase is tetraketide synthase from Cannabis sativa which is also known in the art as olivetol synthase and 3,5,7-trioxododecanoyl-CoA synthase. In one embodiment, tetraketide synthase condenses hexanoyl-CoA with three malonyl-CoA in a multi-step reaction to form 3,5,7-trioxododecanoyl-CoA. In another embodiment, tetraketide synthase condenses butyryl-CoA with three malonyl-CoA in a multi-step reaction to form 3,5,7-trioxodecanoyl-CoA. In another embodiment, the type III polyketide synthase is Steely1 or Steely 2 from Dictyostelium discoideum, comprising a domain with type III polyketide synthase activity, or a variant thereof (e.g., Steely1 (G1516D+G1518A) or Steely1 (G1516R) disclosed in WO/2018/148849). Steely1 is also known in the art as DiPKS or DiPKS1, and Steely2 is also known in the art as DiPKS37.

An olivetolic acid cyclase, as used herein, refers to an enzyme that catalyzes an intramolecular aldol condensation of a 3,5,7-trioxoalkanoyl-CoA to form a 2,4-dihydroxy-6-alkylbenzoic acid, wherein the alkyl group of the benzoic acid contains 1 to 5 carbons. In an embodiment, an olivetolic acid cyclase catalyzes the formation of olivetolic acid from 3,5,7-trioxododecanoyl-CoA. In another embodiment, an olivetolic acid cyclase catalyzes the formation of divarinolic acid from 3,5,7-trioxodecanoyl-CoA. An olivetolic acid cyclase may have the amino acid sequence of SEQ ID NO: 2 or an amino acid sequence with at least 90% identity to SEQ ID NO: 2. Olivetolic acid cyclase from Cannabis sativa is also known in the art as olivetolic acid synthase and 3,5,7-trioxododecanoyl-CoA CoA-lyase.

An aromatic prenyltransferase, as used herein, refers to an enzyme capable of transferring a geranyl diphosphate onto a 5-alkylbenzene-1,3-diol to synthesize a 2-geranyl-5-alkylbenzene-1,3-diol, wherein the alkyl group of the product contains 1 to 5 carbons. In one embodiment, an aromatic prenyltransferase transfers a geranyl disphosphate onto olivetol to synthesize cannabigerol (CBG). In another embodiment, an aromatic prenyltransferase transfers a geranyl disphosphate onto olivetolic acid (OA) to synthesize cannabigerolic acid (CBGA). In another embodiment, an aromatic prenyltransferase transfers a geranyl disphosphate onto divarinolic acid to synthesize cannabigerovarin (CBGV). In another embodiment, an aromatic prenyltransferase transfers a geranyl disphosphate onto divarinolic acid to synthesize cannabigerovarinic acid (CBGVA). An example of an aromatic prenyltransferase is aromatic prenyltransferase from Cannabis sativa which is also known in the art as CsPT1, prenyltransferase 1, geranylpyrophosphate-olivetolic acid geranyltransferase, and geranyl-diphosphate: olivetolate geranytransferase. Further examples of aromatic prenyltransferase include HIPT1 from Humulus lupulus, CsPT4 from Cannabis sativa, and Orf2 (NphB) from Streptomyces Sp. Strain C1190. An aromatic prenyltransferase may have the amino acid sequence of SEQ ID NO: 3, 9, 10 or 11, or an amino acid sequence with at least 90% identity to SEQ ID NO: 3, 9, 10 or 11.

A tetrahydrocannabinolic acid synthase is also known in the art as A9-tetrahydrocannabinolic acid synthase, and synthesizes Δ9-tetrahydrocannabinolic acid by catalyzing the cyclization of the monoterpene moiety in cannabigerolic acid. A tetrahydrocannabinolic acid synthase may have the amino acid sequence of SEQ ID NO:5 or an amino acid sequence with at least 90% identity to SEQ ID NO:5.

A cannabidiolic acid synthase synthesizes cannabidiolic acid by catalyzing the stereoselective oxidative cyclization of the monoterpene moiety in cannabigerolic acid. A cannabidiolic acid synthase may have the amino acid sequence of SEQ ID NO:6 or an amino acid sequence with at least 90% identity to SEQ ID NO:6.

In an embodiment, genetically modified microorganisms provided herein comprise exogenous nucleic acid molecules that encode no more than one, two, or three of hexanoyl-CoA synthetase, type III polyketide synthase (e.g., tetraketide synthase, Steely 1 and Steely 2), and olivetolic acid cyclase; or encode no more than one or two of type III polyketide synthase (e.g., tetraketide synthase, Steely 1 and Steely 2) and olivetolic acid cyclase, and do not comprise exogenous nucleic acid molecules that encode aromatic prenyltransferase, and optionally do not comprise exogenous nucleic acid molecules that encode tetrahydrocannabinolic acid synthase or cannabidiolic acid synthase.

In an embodiment, the nucleic acid molecule comprising nucleic acid sequence encoding at least one of hexanoyl-CoA synthetase comprises amino acid sequence with at least 90%, 95%, 96%, 97%, 98%, 99%, or 100% sequence identity to sequence as shown in SEQ ID NO:4, type III polyketide synthase comprises amino acid sequence with at least 90%, 95%, 96%, 97%, 98%, 99%, or 100% sequence identity to sequence as shown in SEQ ID NO:1, 7 or 8, and olivetolic acid cyclase comprises amino acid sequence with at least 90%, 95%, 96%, 97%, 98%, 99%, or 100% sequence identity to sequence as shown in SEQ ID NO:2. In another embodiment, the nucleic acid molecule does not comprise nucleic acid sequence encoding hexanoyl-CoA synthetase. In another embodiment, the nucleic acid molecule is comprised in a genetically engineered microorganism.

In an embodiment, the nucleic acid molecule comprising nucleic acid sequence encoding type III polyketide synthase comprises amino acid sequence with at least 90%, 95%, 96%, 97%, 98%, 99%, or 100% sequence identity to sequence as shown in SEQ ID NO:1, and olivetolic acid cyclase comprises amino acid sequence with at least 90%, 95%, 96%, 97%, 98%, 99%, or 100% sequence identity to sequence as shown in SEQ ID NO:2.

In an embodiment, the nucleic acid molecule comprising nucleic acid sequence encoding type III polyketide synthase comprises amino acid sequence with at least 90%, 95%, 96%, 97%, 98%, 99%, or 100% sequence identity to sequence as shown in SEQ ID NO:7 or 8.

As used herein, the term “vector” or “nucleic acid vector” means a nucleic acid molecule, such as a plasmid, comprising regulatory elements and a site for introducing transgenic DNA, which is used to introduce said transgenic DNA into a microorganism. The transgenic DNA can encode a heterologous protein, which can be expressed in and isolated from a microorganism. The transgenic DNA can be integrated into nuclear, mitochondrial or chloroplastic genomes through homologous or non-homologous recombination. The transgenic DNA can also replicate without integrating into nuclear, mitochondrial or chloroplastic genomes in an extra-chromosomal vector. The vector can contain a single, operably-linked set of regulatory elements that includes a promoter, a 5′ untranslated region (5′ UTR), an insertion site for transgenic DNA, a 3′ untranslated region (3′ UTR) and a terminator sequence. Vectors useful in the present methods are well known in the art. In one embodiment, the nucleic acid molecule is an episomal vector.

As used herein, the term “episomal vector” refers to a DNA vector based on a bacterial episome that can be expressed in a transformed cell without integration into the transformed cell genome. Episomal vectors can be transferred from a bacteria (e,g, Escherichia coli) to another target microorganism (e.g. a microalgae) via conjugation.

In another embodiment, the vector is a commercially-available vector. As used herein, the term “expression cassette” means a single, operably-linked set of regulatory elements that includes a promoter, a 5′ untranslated region (5′ UTR), an insertion site for transgenic DNA, a 3′ untranslated region (3′ UTR) and a terminator sequence. In an embodiment, the at least one nucleic acid molecule is an episomal vector.

The term “operably-linked”, as used herein, refers to an arrangement of two or more components, wherein the components so described are in a relationship permitting them to function in a coordinated manner. For example, a transcriptional regulatory sequence or a promoter is operably-linked to a coding sequence if the transcriptional regulatory sequence or promoter facilitates aspects of the transcription of the coding sequence. The skilled person can readily recognize aspects of the transcription process, which include, but not limited to, initiation, elongation, attenuation and termination. In general, an operably-linked transcriptional regulatory sequence joined in cis with the coding sequence, but it is not necessarily directly adjacent to it.

The nucleic acid vectors encoding the cannabinoid biosynthetic pathway enzyme therefore contain elements suitable for the proper expression of the enzyme in the microorganism. Specifically, each expression vector contains a promoter that promotes transcription in microorganisms. The term “promoter,” as used herein, refers to a nucleotide sequence that directs the transcription of a gene or coding sequence to which it is operably-linked. Suitable promoters include, but are not limited to, pEF-1 α, p40SRPS8, pH4-1B, py-Tubulin, pRBCMT, pFcpA, pFcpB, pFcpC, pFcpD, HSP70A-RbcS2 (as shown in Table 1 as SEQ ID NO:21-28, 53 and 55; see Slattery et al, 2018), and RbcS2. The skilled person can readily appreciate inducible promoters including chemically-inducible promoters, alcohol inducible promoters, and estrogen inducible promoters can also be used. Predicted promoters, such as those that can be found from genome database mining may also be used. In addition, the nucleic acid molecule or vector may contain one or more introns in front of the cloning site or within a gene sequence to drive a strong expression of the gene of interest. The one or more introns includes introns of FBAC2-1 TUFA-1, EIF6-1, RPS4-1, RbcS2-1, RbcS2-2 (as shown in Table 1 as SEQ ID NO:15-20). The nucleic acid molecule may contain more than one intron or more than one copy of the same intron. The nucleic acid molecule or vector also contains a suitable terminator such as tEF-1a, t40SRPS8, tH4-1B, ty-Tubulin, tRBCMT, tFcpB, tFcpC, tFcpD, tFcpA, tRbcS2 (as shown in Table 1 as SEQ ID NO:29-36, 54 and 56). Seletectable marker genes can also be linked on the vector, such as the kanamycin resistance gene (also known as neomycin phosphotransferase gene II, or nptll), zeocin resistance gene, hygromycin resistance gene, Basta resistance gene, hygromycin resistance gene, or others. As used herein, the term “tag” refers to an amino acid sequence that is recognized by an antibody. The tag amino acid sequence links to, for example, sequence of an enzyme, thereby allowing detection or isolation of the enzyme by the binding between the tag and the tag-specific antibody. For example, common tags known in the art include 6His, MYC, FLAG, V5, HA and HSV. These tags are useful when positioned at the N- or C-terminus.

In an embodiment, the nucleic acid molecule comprises a sequence encoding Rubisco small subunit. Rubisco small subunit may enable the targeting of a polypeptide to which it is attached to be exported to chloroplasts via an internal plastid-targeting signal (Hirakawa and Ishida 2010). Without being bound by theory, it is expected that exporting cannabinoid biosynthetic enzymes to the chloroplast compartment may enhance the exogenous cannabinoid biosynthetic pathway in microalgae because of the availability in the chloroplast of substrates including acetyl-CoA and malonyl-CoA. In some embodiments, the at least one nucleic acid molecule comprises a sequence encoding Rubisco small subunit with an amino acid sequence at least 90%, 95%, 96%, 97%, 98%, 99%, or 100% sequence identity to sequence as shown in SEQ ID NO:12.

As used herein, the term “reporter” refers to a molecule that allows for the detection of another molecule to which the reporter is attached or associated, or for the detection of an organism that comprises the reporter. Reporters can include fluorescent molecules including fluorescent proteins such as green fluorescent protein (GFP), yellow fluorescent protein (YFP), and red fluorescent protein (RFP). In some embodiments, the at least one nucleic acid molecule comprises one or more reporter sequences encoding a reporter with an amino acid sequence with at least 90%, 95%, 96%, 97%, 98%, 99%, or 100% sequence identity to sequence as shown in SEQ ID NO:13-14.

In an embodiment, the nucleic acid molecule or vector encoding the at least one cannabinoid biosynthetic pathway enzyme comprises a promoter nucleic acid sequence selected from SEQ ID NO:21-28, 53 and 55. In another embodiment, the nucleic acid molecule comprises at least one intron sequence selected from SEQ ID NO:15-20. In another embodiment, the nucleic acid molecule comprises a terminator nucleic acid sequence selected from SEQ ID NO:29-36, 54 and 56. In another embodiment, the genetically engineered microorganism comprises a nucleic acid molecule comprising at least one sequence encoding a tag with an amino acid sequence selected from SEQ ID NO:37-42.

The nucleic acid molecule can be constructed to express no more than one, two, or three enzymes associated with the cannabinoid biosynthetic pathway. In an embodiment, the nucleic acid molecule comprises two or more polynucleotide sequences, each of which encodes one cannabinoid biosynthetic pathway enzyme and is operably linked to the same promoter. Where two or three enzymes are encoded in a construct, the construct can contain nucleotide sequence encoding a self-cleaving peptide linker, for example FMDV2a, extFMDV2a, or T2A, which results in the enzymes being produced as separated proteins; or the construct can contain peptide linker sequences linking the enzymes as a fusion protein, for example 3(GGGGS) and FPL1 peptide linker, allowing substrate channelling in which the passing of the intermediary metabolic product of one enzyme directly to another enzyme or active site without its release into solution; or the construct can contain a combination of self-cleaving and non-self-cleaving sequences. In an embodiment, the nucleic acid molecule comprises at least one linker sequence between at least two polynucleotide sequences. In another embodiment, the linker sequence encodes a self-cleaving peptide linker, optionally a self-cleaving peptide linker with an amino acid sequence as shown in SEQ ID NO:43-45. In some embodiments, the at least one nucleic acid molecule comprises one or more linker sequences encoding a peptide linker with an amino acid sequence with at least 90%, 95%, 96%, 97%, 98%, 99%, or 100% sequence identity to sequence as shown in SEQ ID NO:43-48.

In another embodiment, the vector comprises a nucleic acid sequence as described herein. In another embodiment, a host cell is transformed with a vector or nucleic acid molecule comprising a nucleic acid sequence as described herein. In another embodiment, the host cell is any microorganism as described herein.

Nucleic acid sequences as described herein can be provided in vectors in different arrangements or combinations. Each individual sequence that encodes an enzyme of a cannabinoid biosynthetic pathway can be provided in separate vectors. Alternatively, multiple sequences can be provided together in the same vector. For example, nucleic acid sequences encoding a type III polyketide synthase and an olivetolc acid cyclase can be provided together in a first vector, and a nucleic acid sequence encoding a hexanoyl-CoA synthetase can be provided in a second vector. Alternatively, sequences that encode all of the enzymes can be provided together in the same vector. Where more than one sequence that encodes an enzyme is provided in the same vector, the sequences can be provided in separate expression cassettes, or together in the same expression cassette. Where two or more sequences are in the same expression cassette, they can be provided in the same open reading frame so as to produce a fusion protein. Two or more sequences that encode a fusion protein can be separated by linker sequences that encode restriction nuclease recognition sites or self-cleaving peptide linkers. Accordingly, a genetically modified microorganism for the production of cannabinoids can be engineered by stepwise transfection with multiple vectors that each comprises nucleic acid sequences that encode one or more enzymes of a cannabinoid biosynthetic pathway, or with a single vector that comprises nucleic acid sequences that encode all of the enzymes.

As used herein, the term “microalgae” and its derivatives, include photosynthetic and non-photosynthetic microorganisms that are eukaryotes. As used herein, the term “cyanobacteria” and its derivatives, include photosynthetic microorganisms that are prokaryotes. In an embodiment, the microalga is a GC-rich microalga. As used herein, “GC-rich microalga” refers to a microalga wherein the DNA of the nuclear genome and/or the plastid genome comprises at least 50%, at least 55%, at least 60%, at least 65%, at least 70%, or at least 75% GC content. In an embodiment, the microalga is an oleaginous microalga. As used herein “oleaginous” refers to a microalga comprising a lipid conent of at least 35%, at least 40%, at least 45%, or at least 50% by weight. In an embodiment, the microalga is a cold-adapted microalga. As used herein, “cold-adapted” refers to a microalga that grows in temperate, sub-polar, or polar regions in nature, or that has been adapted in artificial growth conditions to grow at temperatures found in temperate, sub-polar, or polar regions. In some embodiments, the cold-adapted microalga grows at a temperature lower than 24° C., lower than 20° C., lower than 16° C., or lower than 12° C. In an embodiment, the microalga is a cold-adapted microalga that exhibits increased lipid content when grown at a temperature lower than 24° C., lower than 20° C., lower than 16° C., or lower than 12° C.

In an embodiment, the microalga is a green alga. In an embodiment, the microalga is from the phylum Chlorophyta. In an embodiment, the microalga is from the genera Ankistrodesmus, Asteromonas, Auxenochlorella, Basichlamys, Botryococcus, Botryokoryne, Borodinella, Brachiomonas, Catena, Carteria, Chaetophora, Characiochloris, Characiosiphon, Chlainomonas, Chlamydomonas, Chlorella, Chlorochytrium, Chlorococcum, Chlorogonium, Chloromonas, Closteriopsis, Dictyochloropsis, Dunaliella, Ellipsoidon, Eremosphaera, Eudorina, Floydiella, Friedmania, Haematococcus, Hafniomonas, Heterochlorella, Gonium, Halosarcinochlamys, Koliella, Lobocharacium, Lobochlamys, Lobomonas, Lobosphaera, Lobosphaeropsis, Marvania, Monoraphidium, Myrmecia, Nannochloris, Oocystis, Oogamochlamys, Pabia, Pandorina, Parietochloris, Phacotus, Platydorina, Platymonas, Pleodorina, Polulichloris, Polytoma, Polytomella, Prasiola, Prasiolopsis, Prasiococcus, Prototheca, Pseudochlorella, Pseudocarteria, Pseudotrebouxia, Pteromonas, Pyrobotrys, Rosenvingiella, Scenedesmus, Spirogyra, Stephanosphaera, Tetrabaena, Tetraedron, Tetraselmis, Trebouxia, Trochisciopsis, Viridiella, Vitreochlamys, Volvox, Volvulina, Vulcanochloris, Watanabea, Yamagishiella, Euglena, Isochrysis, Nannochloropsis. In an embodiment, the microalga is Chlamydomonas reinhardtii, Chlorella vulgaris, Chlorella sorokiniana, Chlorella protothecoides, Tetraselmis chui, Nannochloropsis oculate, Scenedesmus obliquus, Acutodesmus dimorphus, Dunaliella tertiolecta, or Heamatococus plucialis. In another embodiment, the microalga is a diatom, optionally Phaeodactylum tricornutum or Thalassiosira pseudonana.

In another embodiment, the cyanobacterium is from Spirulinaceae, Phormidiaceae, Synechococcaceae, or Nostocaceae. In an embodiment, the cyanobacterium is Arthrospira plantesis, Arthrospira maxima, Synechococcus elongatus, or Aphanizomenon flos-aquae.

The present disclosure also provides a cell culture comprising a genetically engineered microorganism described herein for production of cannabinoids and a medium for culturing the genetically engineered microorganism. In an embodiment, the medium is substantially free of a sugar, i.e., the concentration of the sugar being less than 2%, less than 1.5%, less than 1%, less than 0.5%, or less than 0.1% by weight. In another embodiment, the medium contains no more than trace amounts of a sugar, a trace amount commonly understood in the art as referring to insignificant amounts or amounts near the limit of detection. Sugars known to be required for culturing microorganisms that are not capable of photosynthesis include, but are not limited to, monosaccharides (e.g., glucose, fructose, ribose, xylose, mannose, and galactose) and disaccharides (e.g., sucrose, lactose, maltose, lactulose, trehalose, and cellobiose).

In another embodiment, the medium is substantially free of a fixed carbon source, i.e., the concentration of the fixed carbon source being less than 2%, less than 1.5%, less than 1%, less than 0.5%, or less than 0.1% by weight. In another embodiment, the medium contains no more than trace amounts of a fixed carbon source. The term “fixed carbon source”, as used herein, refers to an organic carbon molecule that is liquid or solid at ambient temperature and pressure that provides a source of carbon for growth, biosynthesis, and/or metabolism. Examples of fixed carbon sources include, but are not limited to, sugars (e.g. glucose, galactose, mannose, fructose, sucrose, lactose), amino acids or amino acid derivatives (e.g. glycine, N-acetylglucosamine, glycerol, floridoside, glucuronic acid, corn starch, depolymerized cellulosic material, plant material (e.g. sugar cane, sugar beet), and carboxylic acid (e.g. hexanoic acid, butyric acid and their respective salts). Sources of fixed carbon are disclosed in WO/2015/168458, the contents of which are herein incorporated by reference.

Microorganisms may be cultured in conditions that are permissive to their growth. It is known that photosynthetic microorganisms are capable of carbon fixation wherein carbon dioxide (which is not a fixed carbon source) is fixed into organic molecules such as sugars using energy from a light source. The fixation of carbon dioxide using energy from a light source is photosynthesis. Suitable sources of light for the provision of energy in photosynthesis include sunlight and artificial lights. Photosynthetic microorganisms are capable of growth and/or metabolism without a fixed carbon source. Microalgae can fix carbon dioxide from a variety of sources, including atmospheric carbon dioxide, industrially-discharged carbon dioxide (e.g. flue gas and flaring gas), and from soluble carbonates (e.g. NaHCO3 and Na2CO3), (see Singh et al 2014, the contents of which are hereby incorporated by reference). A non-fixed carbon source such as carbon dioxide can be added to a culture of microalgae by injection or by bubbling of a carbon dioxide gas mixture into the culture medium. Photosynthetic growth is a form of autotrophic growth, wherein a microorganism is able to produce organic molecules on its own using an external energy source such as light. This is in contrast to heterotrophic growth, wherein a microorganism must consume organic molecules for growth and/or metabolism. Heterotrophic organisms therefore require a fixed carbon source for growth and/or metabolism. Some photosynthetic organisms are capable of mixotrophic growth, wherein the microorganism fixes carbon by photosynthesis while also consuming fixed carbon sources. In mixotrophic growth, the autotrophic metabolism is integrated with a heterotrophic metabolism that oxidizes reduced carbon sources available in the culture medium. Photosynthetic microalgae are commonly cultivated in mixotrophic conditions by adding fixed carbon sources as described herein to the culture medium. Common sources of fixed caron that are used include glucose, ethanol, or waste products from industry such as acetate or glycerol (see Cecchin et al 2018, the contents of which are hereby incorporated by reference). Microorganisms such as microalgae and cyanobacteria may be cultured using methods and conditions known in the art (see, e.g., Biofuels from Algae, eds. Pandey et al., 2014, Elsevier, ISBN 978-0-444-59558-4, the contents of which are hereby incorporated by reference)). Some microorganisms are capable of chemoautotrophic growth, Similar to photosynthetic microorganisms, chemoautotrophic organisms are capable of carbon dioxide fixation but using energy derived from chemical sources (e.g. hydrogen sulfide, ferrous iron, molecular hydrogen, ammonia) rather than light.

Microalgae can be grown in organic conditions without the use of chemicals or additives that contravene the standards for organically-produced products. Microalgae can be grown organically, for example, by growing them in conditions that comply with jurisdictional standards such as the standards set by the United States (US Organic Food Production Act; USDA National Organic Program Certification; USDA Organic Regulations), the European Union (Regulation No 834/2007 prior to Jan. 1, 2021; Regulation 2018/848 from Jan. 1, 2021), and Canada (Canadian Food Inspection Agency Canadian Organic Standards). Growing microalgae in organic conditions permits the production of organic plant natural products in microalgae.

The present disclosure also provides a nucleic acid molecule comprising a nucleotide sequence encoding no more than one, two, or three cannabinoid biosynthetic pathway enzymes. In one embodiment, the nucleic acid molecule comprises nucleic acid sequences encoding no more than one, two, or three of hexanoyl-CoA synthetase, type III polyketide synthase (e.g., tetraketide synthase, Steely 1 and Steely 2), and olivetolic acid cyclase. In another embodiment, the nucleic acid molecule comprises nucleic acid sequences encoding type III polyketide synthase (e.g., tetraketide synthase, Steely 1 and Steely 2), olivetolic acid cyclase, or both, without encoding hexanoyl-CoA synthetase.

The phrase “introducing a nucleic acid molecule into a microorganism” includes both the stable integration of the nucleic acid molecule into the genome of a microorganism to prepare a genetically engineered microorganism as well as the transient integration of the nucleic acid into microorganism. The introduction of a nucleic acid into a cell is also known in the art as transformation. The nucleic acid vectors may be introduced into the microorganism using techniques known in the art including, without limitation, agitation with glass beads, electroporation, agrobacterium-mediated transformation, an accelerated particle delivery method, i.e. particle bombardment, a cell fusion method or by any other method to deliver the nucleic acid vectors to a microorganism.

Particular embodiments of the disclosure include, without limitation, the following:

1. A genetically engineered microorganism that is capable of producing a cannabinoid, wherein the genetically engineered microorganism is a photosynthetic microalga or a cyanobacterium, and wherein the genetically engineered microorganism does not comprise an exogenous nucleic acid molecule encoding aromatic prenyltransferase.
2. The genetically engineered microorganism of embodiment 1, which is capable of producing tetrahydrocannabinolic acid or tetrahydrocannabinol, and does not comprise an exogenous nucleic acid molecule encoding tetrahydrocannabinolic acid synthase.
3. The genetically engineered microorganism of embodiment 1 or 2, which is capable of producing cannabidiolic acid or cannabidiol, and does not comprise an exogenous nucleic acid molecule encoding cannabidiolic acid synthase.
4. The genetically engineered microorganism of any one of embodiments 1 to 3, wherein the genetically engineered microorganism comprises at least one exogenous nucleic acid molecule that encodes tetraketide synthase and olivetolic acid cyclase.
5. The genetically engineered microorganism of embodiment 4, wherein the tetraketide synthase comprises amino acid sequence with at least 90% sequence identity to sequence as shown in SEQ ID NO:1, and the olivetolic acid cyclase comprises amino acid sequence with at least 90% sequence identity to sequence as shown in SEQ ID NO:2.
6. The genetically engineered microorganism of embodiment 4 or 5, wherein the at least one exogenous nucleic acid molecule comprises a first polynucleotide sequence encoding tetraketide synthase and a second polynucleotide sequence encoding olivetolic acid cyclase.
7. The genetically engineered microorganism of embodiment 6, wherein the first polynucleotide sequence is 5′ to the second polynucleotide sequence.
8. The genetically engineered microorganism of embodiment 6 or 7, wherein the at least one exogenous nucleic acid molecule further comprises at least one linker sequence between the first and second polynucleotide sequences.
9. The genetically engineered microorganism of embodiment 8, wherein the linker sequence encodes a self-cleaving linker sequence (e.g., amino acid sequence SEQ ID NO:43-45) or a fusion linker sequence (e.g., amino acid sequence SEQ ID NO:46-48).
10. The genetically engineered microorganism of any one of embodiments 6 to 9, wherein the first polynucleotide sequence comprises at least one intron sequence (e.g., SEQ ID NO:15-20).
11. The genetically engineered microorganism of embodiment 4 or 5, wherein the at least one exogenous nucleic acid molecule comprises a first nucleic acid molecule encoding tetraketide synthase and a second nucleic acid molecule encoding olivetolic acid cyclase.
12. The genetically engineered microorganism of any one of embodiments 4 to 11, wherein the at least one exogenous nucleic acid molecule further comprises one or more of a promoter nucleic acid sequence (e.g., SEQ ID NO:21-28, 53 and 55), a sequence encoding a tag (e.g., amino acid sequence SEQ ID NO:37-42), a sequence encoding a reporter (e.g., amino acid sequence SEQ ID NO:13-14), a sequence encoding Rubisco small subunit (e.g., amino acid sequence SEQ ID NO:12), and a terminator nucleic acid sequence (e.g., SEQ ID NO:29-36, 54 and 56).
13. The genetically engineered microorganism of any one of embodiments 4 to 12, wherein the at least one exogenous nucleic acid molecule is an episomal vector.
14. The genetically engineered microorganism of any one of embodiments 4 to 13, wherein the genetically engineered microorganism consists of the at least one exogenous nucleic acid molecule.
15. The genetically engineered microorganism of any one of embodiments 1 to 3, wherein the genetically engineered microorganism comprises at least one exogenous nucleic acid molecule that encodes Steely1, Steely 2, or a variant thereof.
16. The genetically engineered microorganism of embodiment 15, wherein the variant of Steely1 or Steely2 comprises amino acid sequence with at least 90% sequence identity to sequence as shown in SEQ ID NO:7 or SEQ ID NO:8, respectively.
17. The genetically engineered microorganism of embodiment 15 or 16, wherein the at least one exogenous nucleic acid molecule comprises at least one intron sequence (e.g., SEQ ID NO:15-20).
18. The genetically engineered microorganism of any one of embodiments 15 to 17, wherein the at least one exogenous nucleic acid molecule further comprises one or more of a promoter nucleic acid sequence (e.g., SEQ ID NO:21-28, 53 and 55), a sequence encoding a tag (e.g., amino acid sequence SEQ ID NO:37-42), a sequence encoding a reporter (e.g., amino acid sequence SEQ ID NO:13-14), a sequence encoding Rubisco small subunit (e.g., amino acid sequence SEQ ID NO:12), and a terminator nucleic acid sequence (e.g., SEQ ID NO:29-36, 54 and 56).
19. The genetically engineered microorganism of any one of embodiments 15 to 18, wherein the at least one exogenous nucleic acid molecule is an episomal vector.
20. The genetically engineered microorganism of any one of embodiments 15 to 19, wherein the genetically engineered microorganism consists of the at least one exogenous nucleic acid molecule.
21. The genetically engineered microorganism of any one of embodiments 1 to 20, wherein the genetically engineered microorganism does not comprise an exogenous nucleic acid molecule encoding hexanoyl-CoA synthetase.
22. The genetically engineered microorganism of any one of embodiments 1 to 21, wherein the microalga is a diatom or a Chlorophyta.
23. The genetically engineered microorganism of embodiment 22, wherein the microalga is Phaeodactylum tricornutum or Thalassiosira pseudonana.
24. The genetically engineered microorganism of embodiment 23, wherein the microalga is Phaeodactylum tricornutum.
25. The genetically engineered microorganism of embodiment 22, wherein the microalga is Chlamydomonas reinhardtii or Chlorella vulgaris.
26. The genetically engineered microorganism of embodiment 25, wherein the microalga is Chlamydomonas reinhardtii.
27. The genetically engineered microorganism of any one of embodiments 1 to 21, wherein the cyanobacterium is a Spirulinaceae, Phormidiaceae, Synechococcaceae, or Nostocaceae, optionally Arthrospira plantesis, Arthrospira maxima, Synechococcus elongatus or Aphanizomenon flos-aquae.
28. A cell culture comprising the genetically engineered microorganism of any one of embodiments 1 to 27, and a medium that is substantially free of a sugar.
29. The cell culture of embodiment 28, wherein the sugar is present in the medium at a concentration of less than 2% by weight.
30. The cell culture of embodiment 29, wherein the sugar is present in the medium at a concentration of less than 1% by weight.
31. The cell culture of embodiment 30, wherein the sugar is present in the medium at a concentration of less than 0.5% by weight.
32. The cell culture of embodiment 31, wherein the sugar is present in the medium at a concentration of less than 0.1% by weight.
33. The cell culture of embodiment 32, wherein the sugar is present in the medium at no more than trace amounts.
34. The cell culture of any one of embodiments 28 to 33, wherein the sugar is a monosaccharide.
35. The cell culture of embodiment 34, wherein the monosaccharide is at least one of glucose, fructose, ribose, xylose, mannose, and galactose.
36. The cell culture of any one of embodiments 28 to 33, wherein the sugar is a disaccharide.
37. The cell culture of embodiment 36, wherein the disaccharide is at least one of sucrose, lactose, maltose, lactulose, trehalose, and cellobiose.
38. The cell culture of any one of embodiments 28 to 37, wherein the medium is substantially free of a fixed carbon source.
39. The cell culture of embodiment 38, wherein the fixed carbon source is at least one of carboxylic acid and glycerol.
40. The cell culture of embodiment 39, wherein the carboxylic acid is hexanoic acid.
41. The cell culture of any one of embodiments 28 to 40, wherein the cell culture undergoes autotrophic growth.
42. The cell culture of embodiment 41, wherein the autotrophic growth is photosynthetic growth.
43. The cell culture of embodiment 42, wherein the photosynthetic growth occurs in the presence of a solar light source.
44. The cell culture of embodiment 42, wherein the photosynthetic growth occurs in the presence of an artificial light source.
45. The cell culture of any one of embodiments 28 to 44, wherein the cell culture undergoes growth in organic conditions.
46. A method for producing a cannabinoid in a genetically engineered microorganism, comprising introducing into the microorganism at least one nucleic acid molecule encoding tetraketide synthase and olivetolic acid cyclase, wherein the microorganism is a microalga or a cyanobacterium.
47. A method for producing a cannabinoid in a genetically engineered microorganism, comprising introducing into the microorganism at least one nucleic acid molecule encoding Steely1, Steely 2, or a variant thereof, wherein the microorganism is a microalga or a cyanobacterium.
48. A method for producing a cannabinoid in a wild type microorganism, comprising culturing the microorganism in a medium comprising a 2,4-dihydroxy-6-alkylbenzoic acid or a 2,4-dihydroxy-6-alkylbenzoate, wherein the microorganism is a microalga or a cyanobacterium.

The following non-limiting Examples are illustrative of the present disclosure:

### Example 1

Episomal vectors construction and diatom Phaeodactylum tricornutum cells transformation

### Example 1.1 Gene Sequences

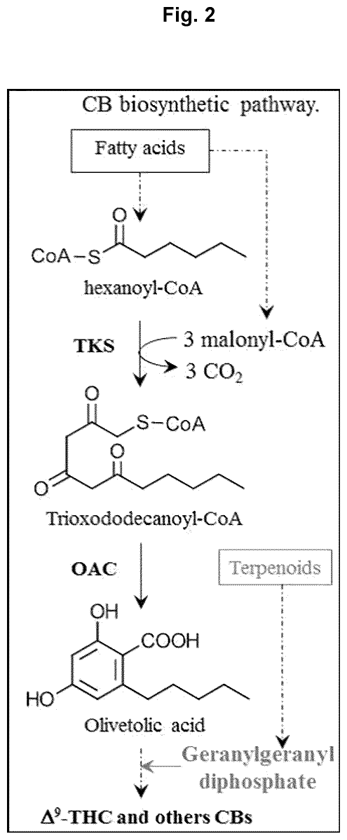

It has been suggested that hexanoyl-CoA synthetase converts hexanoic acid to hexanoyl-CoA early in CB biosynthetic pathway (FIG. 1; modified from Gagne et al 2012). Another early metabolite intermediate in the CB biosynthetic pathway is olivetolic acid (OA) that forms the polyketide skeleton of cannabinoids. Without wishing to be bound by theory, OA is produced as follows (FIG. 2): First, a type III tetra/polyketide synthase (TKS) enzyme condenses hexanoyl-CoA with three malonyl-CoA in a multi-step reaction to form trioxododecanoyl-CoA. Then, the olivetolic acid cyclase (OAC) catalyzes an intramolecular aldol condensation to yield OA. In subsequent steps, CB diversification is generated by the sequential action of “decorating” enzymes on the OA backbone, which leads to cannabinoids Δ9-tetrahydrocannabinolic acid (THCA) and cannabidiolic acid (CBDA), each of which decarboxylates to yield Δ9-tetrahydrocannabinol (THC) and cannabidiol (CBD), respectively (FIG. 1).

The gene sequence for TKS and OAC have been identified and characterized in vitro (Lussier 2012; Gagne et al 2012; Marks et al 2009; Stout et al 2012; Taura et al 2009). The complete coding sequences for non-optimized TKS (GenBank: AB164375.1) and OAC (GenBank: JN679224.1) were obtained from public databases. The open reading frame of TKS (1158 bp) encodes for a protein of 385 amino acids with a calculated MW of 42 kDa (Taura et al 2009; Flores-Sanchez et al 2010). Whereas OAC is a relatively small sequence (485 bp) encoding for a small protein of 101 amino acids and a MW of 12 kDa (Marks et al 2009). Without wishing to be bound by theory, codon optimization is suggested to improve protein expression in a host organism by replacing the nucleic acids coding for a particular amino acid (i.e. a codon) with another codon which is purportedly better expressed in the host organism. This effect may arise due to different organisms showing preferences for different codons. In particular, microalgae and cyanobacteria may prefer different codons from plants and animals. The process of altering the sequence of a nucleic acid to achieve better expression based on codon preference is called codon optimization. Statistical methods have been generated to analyze codon usage bias in various organisms and many computer algorithms have been developed to implement these statistical analyses in the design of codon optimized gene sequences (Lithwick and Margalit 2003). Other modifications in codon usage to increase protein expression that are not dependent on codon bias have also been described (Welch et al 2009). The open reading frame of constructs comprising genes and other elements (e.g., reporters, tags, peptide linkers) was codon-optimized (e.g., SEQ ID NO:49-52), synthesized, and inserted into transformation vectors.

### Example 1.2 Engineered Diatoms

Microalgae provide a promising but challenging platform for the bioproduction of high value chemicals. Compared with model organisms such as Escherichia coli and Saccharomyces cerevisiae, characterization of the complex biology and biochemistry of algae and strain improvement has been hampered by inefficient molecular tools. To date, many microalgae are transformable but the introduced DNA is integrated randomly into the nuclear genome by mechanisms involving non-homologous recombination, and the chance to encounter gene silencing is high. Hence, molecular tools to circumvent these challenges are necessary to facilitate efficient genetic engineering. Recently, an episomal vector system for diatoms was developed and shown to be highly stable (Karas et al 2015). Since episomes should not be affected by gene silencing mechanism, a diatom strain was engineered with constructs comprising TKS and OAC transgenes. Constructs optimized for the codon usage of Phaeodactylum tricornutum are shown in SEQ ID NO:49-52. These optimized sequences can also be used for other diatoms such as Thalassiosira pseudonana.

### Example 1.3 Extraction for HPLC Analysis

HPLC analysis was conducted on cell extracts produced by the exemplary method described herein. Approximately 100 mg of algal culture was centrifuged, and the supernatant was discarded. 5 ml of 100% ethanol was added to the pellet and kept at −20° C. overnight. The pellet was centrifuged at 4° C. for 10 min at 4000 g. 1 ml of the supernatant was transferred to 1.7 ml Eppendorf tubes and the ethanol was evaporated in a Speedvac at maximum vacuum level and no heating. 250 μl of mobile phase solution (Water:formic acid:acetonitrile in 59.9%, 0.1% and 40%) was used to resuspend the pellet. The suspension was homogenised by vortexing each tube for 2 minutes at high speed, then centrifuged at 4° C. for 10 min at maximum speed. 200 μl of the supernatant was collected in an HPLC vial.

### Example 1.4 HPLC Analysis

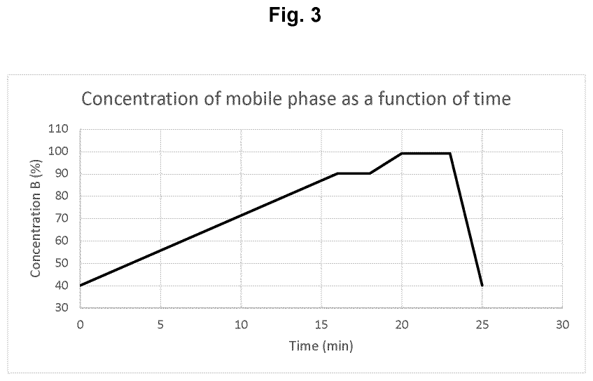

Instrument: Prominence-i LC-2030 C 3D; Detector: UV-DAD//PDA; Column: P/No: OOF-4633-EVO, Model Kinetex 5 μm EVO C18 100 Å, LC Column 150×4.6 mm, Serial No: H15010692, B/No: 5720-050; Oven temperature: 30° C.; Flow: 0.5 mL/min; Mobile phase: A=water with 1% formic acid, B=Acetonitrile 100%; Gradient phase: TO: 40% B, T16: 90% B, T18: 90% B, T20: 99% B, T23: 99% B, T25: 40% B. The concentration of the mobile phase as a function of time is shown in FIG. 3.

The analyzed wavelength was chose based on the maximal peak of each standard in a wild type algae matrix in a Cary 60 UV-Vis precision spectrophotometer: THC: 280 nm, 17.8-18.4 min depending on the neutral or acid form (THCA will appear further); CBD: 275 nm, 15.09-15.4 min; CBN: 285 nm, 17-17.5 min.

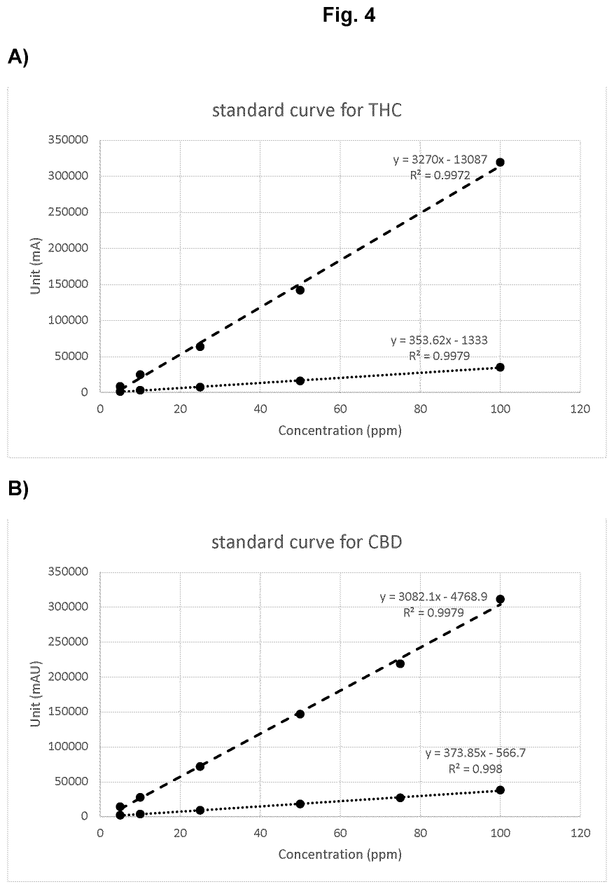

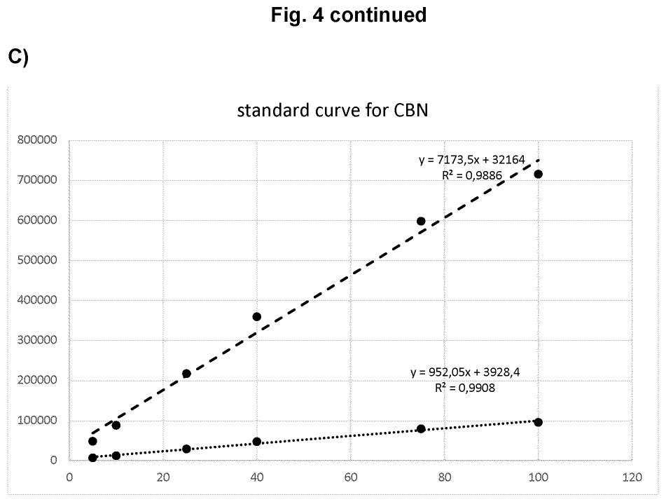

Each standard was diluted into 0, 5, 10, 25, 50, 75 and 100 ppm concentration in a solvent made of wild type P.tricornutum extract as a matrix to determine THC, CBD, or CBN peaks above background in samples. Peaks in samples were identified after normalization with the standard curve and the blank. Standard curves for THC, CBD, and CBN are shown in FIG. 4.

### Example 2

### Example 2.1 (AC 1/Ptref1)

A construct comprising sequences that encode TKS and OAC enzymes was transformed into P.tricornutum.

A construct (Ptref1, SEQ ID NO:49) comprising from 5′ to 3′: a TKS-encoding sequence (position 1 to 1155); a T2A self-cleaving peptide linker sequence (position 1156 to 1218); and an OAC-encoding sequence (position 1219 to 1521) was inserted into a modified pPtGE30 plasmid (Slattery et al 2018) containing a Zeocin resistance gene for algae and a Chloremphenicol resistance gene for E. coli and His selection in yeast.

The construct was operably linked to a 40SRPS8 promoter (SEQ ID NO:22) and a FcpA terminator (SEQ ID NO:54).

The PtGE30 episomal vector was conjugated to P.tricornutum from E. coli.

A Zeocin-resistant clone of P.tricornutum was verified by PCR and full episome sequencing, and selected for analysis by HPLC.

### Example 2.2 (AC 2/Ptref2)

A construct comprising sequences that encode TKS and OAC enzymes was transformed into P.tricornutum.

A construct (Ptref2, SEQ ID NO:50) comprising from 5′ to 3′: a TKS-encoding sequence (position 1 to 1155); a 3(GGGGS) peptide linker sequence (position 1156 to 1200); and an OAC-encoding sequence (position 1201 to 1503) was inserted into a modified pPtGE30 plasmid (Slattery et al 2018) containing a Zeocin resistance gene for algae and a Chloremphenicol resistance gene for E. coli and His selection in yeast.

The construct was operably linked to a 40SRPS8 promoter (SEQ ID NO:22) and a FcpA terminator (SEQ ID NO:54).

The PtGE30 episomal vector was conjugated to P.tricornutum from E. coli.

A Zeocin-resistant clone of P.tricornutum was verified by PCR and full episome sequencing, and selected for analysis by HPLC.

### Example 2.3 (AC 3/Ptref3)

A construct comprising sequences that encode TKS and OAC enzymes was transformed into P.tricornutum.

A construct (Ptref3, SEQ ID NO:51) comprising from 5′ to 3′: a TKS-encoding sequence (position 1 to 1155); a 6His tag (position 1156 to 1173); a T2A self-cleaving peptide linker sequence (position 1174 to 1236); an OAC-encoding sequence (position 1237 to 1539); and a Myc tag sequence (position 1540 to 1569) was inserted was inserted into a modified pPtGE30 plasmid (Slattery et al 2018) containing a Zeocin resistance gene for algae and a Chloremphenicol resistance gene for E. coli and His selection in yeast.

The construct was operably linked to a 40SRPS8 promoter (SEQ ID NO:22) and a FcpA terminator (SEQ ID NO:54).

The PtGE30 episomal vector was conjugated to P.tricornutum from E. coli.

A Zeocin-resistant clone of P.tricornutum was verified by PCR and full episome sequencing, and selected for analysis by HPLC.

### Example 2.4 (AC 7/Ptref7)

A construct comprising sequences that encode TKS and OAC enzymes was transformed into P.tricornutum.

A construct (Ptref7, SEQ ID NO:52) comprising from 5′ to 3′: a YFP reporter sequence (position 1 to 753); a glycine codon (position 754 to 756); a TKS-encoding sequence (position 757 to 1911); a 3(GGGGS) peptide linker sequence (position 1912 to 1956); an OAC-encoding sequence (position 1957 to 2259); and a Myc tag sequence (position 2260 to 2289) was inserted was inserted into a modified pPtGE30 plasmid (Slattery et al 2018) containing a Zeocin resistance gene for algae and a Chloremphenicol resistance gene for E. coli and His selection in yeast.

The construct was operably linked to a 40SRPS8 promoter (SEQ ID NO:22) and a FcpA terminator (SEQ ID NO:54).

The PtGE30 episomal vector was conjugated to P.tricornutum from E. coli.

A Zeocin-resistant clone of P.tricornutum was verified by PCR and full episome sequencing, and selected for analysis by HPLC.

### Example 2.5

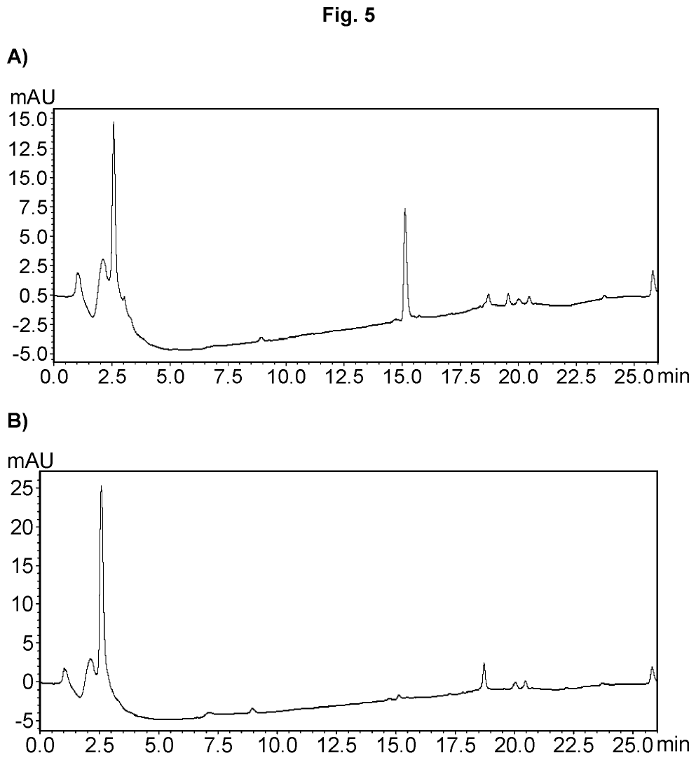

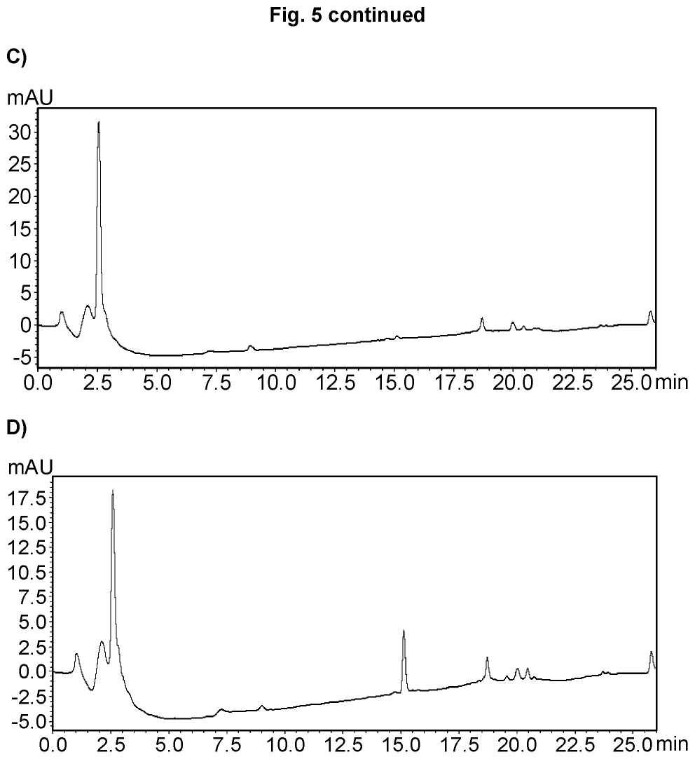

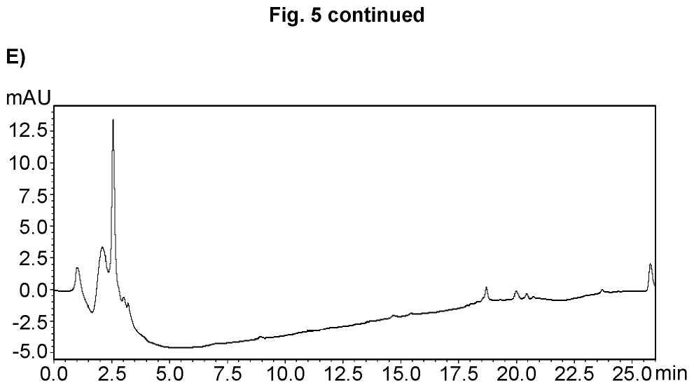

HPLC curves indicated the presence of CBD, THC, and other cannabinoids in clones of P.tricornutum transformed with constructs Ptref1 (FIG. 5A), Ptref2 (FIG. 5B), Ptref3 (FIG. 5C), and Ptref7 (FIG. 5D) comprising TKS and OAC transgenes, as compared to a wild type control (FIG. 5E). This result was unexpected, as it was believed that additional exogenous enzymes including APT and CBDAS/THCAS would need to be transformed into a microorganism to complete the cannabinoid biosynthetic pathway.

The amount of CBD in each sample was calculated based on the standard curve for CBD detected by HPLC (FIG. 4B) and are shown in Table 2:

TABLE 2
Quantification of CBD in transformed P. tricornutum

| | CBD in sample volume | mg of CBD in g of |
|---|---|---|
| Sample | (ppm) | algae |
| Ptref1 | 10.30982123 | 0.515491061 |
| Ptref2 | 2.77242789 | 0.138621395 |
| Ptref3 | 2.671847117 | 0.133592356 |
| Ptref7 | 17.80146653 | 0.890073327 |
| Wild Type Control | 0 | 0 |

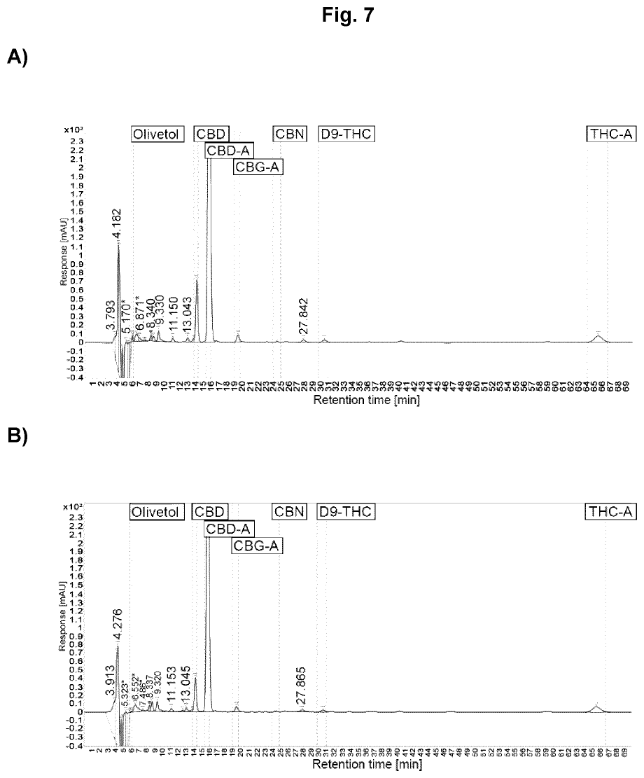

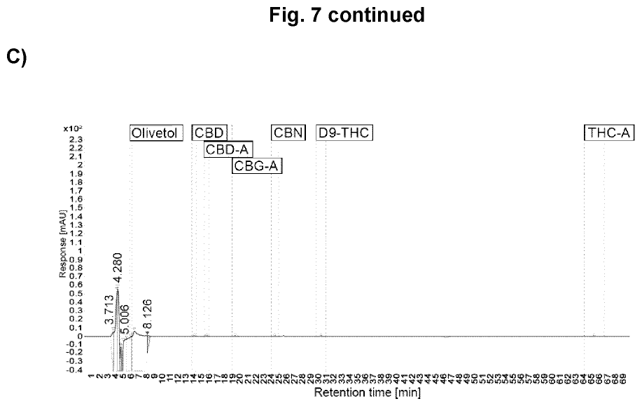

Analysis by UPLC further indicated the presence of CBD and THC in clones of P.tricornutum transformed with constructs Ptref1 (FIG. 7A) and Ptref2 (FIG. 7B) as compared to a wild-type control (FIG. 7C).

These results indicate that a microalga can produce cannabinoids when transformed only with the genes that encode for TKS and OAC enzymes, indicating that microalgae might have enzymes that utilize Olivetolic Acid (OA), and/or derivatives, as a substrate as well as enzymes that are able to synthesize CBD and other cannabinoids.

### Example 3

Vector construction and Chlorophyta Chlamydomonas reinhardtii transformation.

### Example 3.1 Plasmid Growth and Extraction from E. coli

Synthetic constructs for transformation into C.reinhardtii were first inserted into a default vector (KanR, high copy) and transformed into E. coli by electroporation. Transformed E. coli was grown to bulk the plasmids containing the constructs. Positive E. coli were confirmed by colony PCR. The plasmids were then extracted and prepared for Gibson assembly into pChlamy3. pChlamy3 contains the strong hybrid promoter HSP70-RbcS2 and the intron 1 of RbcS2 in front of the cloning site.

### Example 3.2 Gibson Assembly and Transformation in E. coli

Assembled pChlamy3 vectors were used to transform E. coli by heat shock. Positive colonies were grown on ampicillin plates and confirmed by colony PCR. Transformed E. coli was then grown in liquid media LB-amp100 to bulk the vector before extraction and purification (Biobasic, miniprep kit). After linearization (digestion with ScaI) for 3h, linearized vectors were verified on agarose gel 1% and purified (Biobasic, PCR clean up kit). Purified vectors were used for the transformation of C. reinhardtii cells.

### Example 3.3 Transformation of C.Reinhardtii by Electroporation

Cells were cultivated mixotrophically at 25° C. in Tris-acetate phosphate (TAP) medium under moderate and continuous white fluorescent light at the intensity of 50 μmol photons m⁻² s⁻¹ in shake flasks or on agar plates with relative humidity (Rh) of 50%.

Electroporation was performed for transformation as described previously (Shimogawara et al. 1998; Wittkopp 2018; Wang et al. 2019) with slight modifications. C. reinhardtii cells were transformed using the Bio-Rad Genepulser Xcell™ electroporation machine and 4 mm cuvette under the following parameters: voltage 0.5 kV; capacitance 50 μF; resistance 800Ω.

Briefly, liquid state cells were grown in 30 mL TAP culture medium in a 125 mL Erlenmeyer flask with an initial OD₇₅₀ₙₘ of 0.1 (1×10⁵ cells/mL) with gentle shaking (100 rpm) to a final O.D₇₅₀ₙₘ of 0.7 (7×10⁶ cells/mL). Cells were harvested by centrifugation at 7000×g for 5 min and then washed three times by resuspending the pellet in 5 mL of Max Efficiency™ Transformation Reagent for Algae (Invitrogen Cat no #A24229) and centrifuged in the same conditions as in the harvesting step. The sample were incubated on ice for 10 min prior to electroporation which was performed by applying an electric pulse using 250 μL of C. reinhardtii cells and 500 ng of linearized purified plasmid. Transgenic strains were resuspended in 5 mL of TAP liquid medium supplemented with 40 mM sucrose (TAP/sucrose) and then incubated at 25° C. with gentle shaking (100 rpm) for 22 h under continuous light. After incubation, the transformed cells were harvested by centrifugation at 7000×g for 5 min and resuspended in 250 μL of Max Efficiency. Then, spread on TAP agar media supplemented with Hygromycin (10 μg/mL) and incubated in a growth chamber for around 5 to 7 days.

When single clones appeared on agar Petri dish, total number of transformants on each plate was counted using OpenCFU software to determine transformation efficiency.

### Example 3.3 Transgenic Construct and HPLC Analysis

A construct comprising sequences that encode TKS and OAC enzymes was transformed into C.reinhardtii. The construct (G1C1, SEQ ID NO:57) comprising from 5′ to 3′: a TKS-encoding sequence (position 1 to 1155); a FMDV2A self-cleaving peptide linker sequence (position 1156 to 1227); and an OAC-encoding sequence (position 1228 to 1530) was inserted into pChlamy3 plasmid. The construct was operably linked to a HSP70A-RbcS2 Hybrid promoter (SEQ ID NO:55) and a RbcS2 terminator (SEQ ID NO:56). The vector was transfected into C.reinhardtii strain C-137 by electroporation.

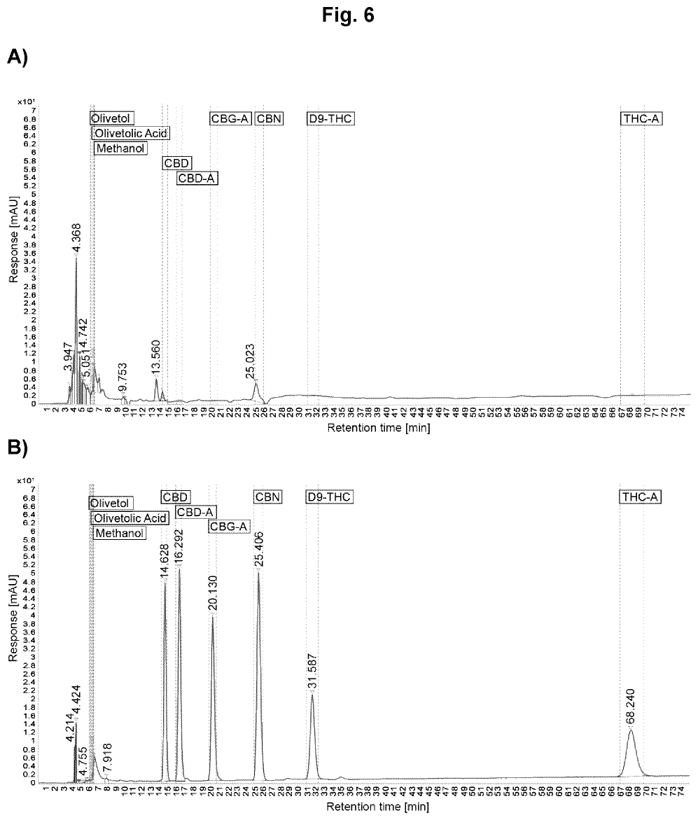

A positive transformant was selected by hygromycin resistance and PCR, and grown in TAP media before harvesting and extracting for analysis by UPLC. UPLC analysis at 220 nm revealed the presence of a peak at 25.023 min (FIG. 6A) that corresponds to Cannabinol (CBN) at 25.406 min in the control sample with cannabinoid standards (FIG. 6B). The control sample with cannabinoid standards (FIG. 6B) shows the THCA peak at 68.240, the THC peak at 31.587 min, the CBN peak at 25.406 min, the CBGA peak at 20.130 min, the CBDA peak at 16.292 min, and the CBD peak at 14.628 min.

While the present disclosure has been described with reference to what are presently considered to be the preferred example, it is to be understood that the disclosure is not limited to the disclosed Examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCES

Altschul, S. F. et al (1990) Basic local alignment search tool.” Journal of molecular biology 215, 403-410.
Altschul, S. F. et al (1997) Gapped BLAST and PSI-BLAST: a new generation of protein database search programs. Nucleic acids research 25, 3389-3402.
Cecchin, M. et al. (2018) Molecular basis of autotrophic vs mixotrophic growth in Chlorella sorokiniana. Sci Rep, 8(1):6465.
DeLoache, W. C., et al. (2015) An enzyme-coupled biosensor enables (S)-reticuline production in yeast from glucose. Nat Chem Biol, 11(7):465.
Diaz-Santos, E., et al. (2013) Efficiency of different heterologous promoters in the unicellular microalga Chlamydomonas reinhardtii. Biotechnol Prog, 29(2):319-328.
ElSohly, M. A. and Slade D. (2005) Chemical constituents of marijuana: The complex mixture of natural cannabinoids. Life Sciences, 2005. 78(5):539-548.
Flores-Sanchez, I. J., et al. (2010) In silicio expression analysis of PKS genes isolated from Cannabis sativa L. Genet Mol Biol, 33(4): 703-713.
Fossati, E., et al. (2014) Reconstitution of a 10-gene pathway for synthesis of the plant alkaloid dihydrosanguinarine in Saccharomyces cerevisiae. Nat Commun, (5):3283.
Gagne, S. J., et al. (2012) Identification of olivetolic acid cyclase from Cannabis sativa reveals a unique catalytic route to plant polyketides. Proc Natl Acad Sci USA 109(31):12811-12816.
Gibson, D. G., et al. (2009) Enzymatic assembly of DNA molecules up to several hundred kilobases. Nat Meth, 6(5):343-345.
Hirakawa, Y., and Ishida, K. (2010) Internal plastid-targeting signal found in a RubisCO small subunit protein of a chlorarachniophyte alga. Plant J, 64(3), 402-410.
Karas, B. J., et al., Designer diatom episomes delivered by bacterial conjugation. Nat Commun, 2015. 6: p. 6925.
Karlin, S. and Altschul, S. F. (1990) Methods for assessing the statistical significance of molecular sequence features by using general scoring schemes. Proceedings of the National Academy of Sciences 87, 2264-2268.
Karlin, S. and Altschul, S. F. (1993) Applications and statistics for multiple high-scoring segments in molecular sequences. Proceedings of the National Academy of Sciences 90, 5873-5877.
Keasling, J. D. (2012) Synthetic biology and the development of tools for metabolic engineering. Metab Eng, 14(3):189-195.
Lithwick G, and Margalit H. (2003) Hierarchy of sequence-dependent features associated with prokaryotic translation. Genome Research 13:2665-2673.
Lussier, F. X., et al. (2012) Engineering microbes for plant polyketide biosynthesis. Comput Struct Biotechnol J 3:e201210020.
Marks, M. D., et al. (2009) Identification of candidate genes affecting Delta9-tetrahydrocannabinol biosynthesis in Cannabis sativa. J Exp Bot, 60(13):3715-3726.
Pandey, A. et al. (2013) Biofuels from Algae 1^{st} Ed., Elsevier, ISBN 9780444595584.
Plecenikova, A., et al. (2013) Studies on recombination processes in two Chlamydomonas reinhardtii endogenous genes, NIT1 and ARG7. Protist, 164(4):570-582.
Schroda, M., et al. (2000) The HSP70A promoter as a tool for the improved expression of transgenes in Chlamydomonas. Plant J, 2000. 21(2): 121-131.
Shimogawara, K., et al. (1998) High-efficiency transformation of Chlamydomonas reinhardtii by electroporation. Genetics, 148: 1821-1828.
Singh, N. D., et al. (2009) Chloroplast-derived vaccine antigens and biopharmaceuticals: protocols for expression, purification, or oral delivery and functional evaluation. Methods Mol Biol, 483:163-192.
Singh, S. P., and Singh, P. (2014) Effect of C02 concentration on algal growth: A review. Ren Sus En Rev, 38:172-179.
Slattery, S. S., et al. (2018) An expanded plasmid-based genetic toolbox enables Cas9 genome editing and stable maintenance of synthetic pathways in Phaeodactylum tricornutum. ACS Synthetic Biology, 7(2):328-338.
Stout, J. M., et al. (2012) The hexanoyl-CoA precursor for cannabinoid biosynthesis is formed by an acyl-activating enzyme in Cannabis sativa trichomes. Plant J, 71(3):353-365.
Taura, F., et al. (2009) Characterization of olivetol synthase, a polyketide synthase putatively involved in cannabinoid biosynthetic pathway. FEBS Lett, 583(12):2061-2066.
Wang, L., et al. (2019) Rapid and high efficiency transformation of Chlamydomonas reinhardtii by square-wave electroporation. Bioscience Reports, 39.
Welch et al. (2009) Design parameters to control synthetic gene expression in Escherichia coli. PLoS ONE 4: e7002.
Wittkopp, T. M. (2018) Nuclear Transformation of Chlamydomonas reinhardtii by electroporation, Bio-Protocol, 8.

1. A genetically engineered microorganism that is capable of producing a cannabinoid, wherein the genetically engineered microorganism is a photosynthetic microalga or a cyanobacterium, and wherein the genetically engineered microorganism does not comprise an exogenous nucleic acid molecule encoding aromatic prenyltransferase.

2. The genetically engineered microorganism of claim 1, which is capable of producing tetrahydrocannabinolic acid or tetrahydrocannabinol, and does not comprise an exogenous nucleic acid molecule encoding tetrahydrocannabinolic acid synthase.

3. The genetically engineered microorganism of claim 1, which is capable of producing cannabidiolic acid or cannabidiol, and does not comprise an exogenous nucleic acid molecule encoding cannabidiolic acid synthase.

4. The genetically engineered microorganism of claim 1 wherein the genetically engineered microorganism comprises at least one exogenous nucleic acid molecule that encodes tetraketide synthase and olivetolic acid cyclase.

5. The genetically engineered microorganism of claim 4, wherein the tetraketide synthase comprises amino acid sequence with at least 90% sequence identity to sequence as shown in SEQ ID NO:1, and the olivetolic acid cyclase comprises amino acid sequence with at least 90% sequence identity to sequence as shown in SEQ ID NO:2.

6. The genetically engineered microorganism of claim 4, wherein the at least one exogenous nucleic acid molecule comprises a first polynucleotide sequence encoding tetraketide synthase and a second polynucleotide sequence encoding olivetolic acid cyclase.

7. The genetically engineered microorganism of claim 6, wherein the first polynucleotide sequence is 5′ to the second polynucleotide sequence.

8. The genetically engineered microorganism of claim 6, wherein the at least one exogenous nucleic acid molecule further comprises at least one linker sequence between the first and second polynucleotide sequences.

9. The genetically engineered microorganism of claim 4, wherein the at least one exogenous nucleic acid molecule comprises a first nucleic acid molecule encoding tetraketide synthase and a second nucleic acid molecule encoding olivetolic acid cyclase.

10. The genetically engineered microorganism of claim 1 wherein the genetically engineered microorganism comprises at least one exogenous nucleic acid molecule that encodes Steely1, Steely 2, or a variant thereof.

11. The genetically engineered microorganism of claim 10, wherein the variant of Steely1 or Steely2 comprises amino acid sequence with at least 90% sequence identity to sequence as shown in SEQ ID NO:7 or SEQ ID NO:8, respectively.

12. The genetically engineered microorganism of claim 4, wherein the at least one exogenous nucleic acid molecule is an episomal vector.

13. The genetically engineered microorganism of claim 4, wherein the genetically engineered microorganism consists of the at least one exogenous nucleic acid molecule.

14. The genetically engineered microorganism of claim 1, wherein the genetically engineered microorganism does not comprise an exogenous nucleic acid molecule encoding hexanoyl-CoA synthetase.

15. The genetically engineered microorganism of claim 1, wherein the microalga is a diatom or a Chlorophyta.

16. The genetically engineered microorganism of claim 15, wherein the microalga is Phaeodactylum tricornutum or Chlamydomonas reinhardtii.

17. A cell culture comprising the genetically engineered microorganism of claim 1, and a medium that is substantially free of a sugar.

18. The cell culture of claim 17, wherein the medium is substantially free of a fixed carbon source.

19. The cell culture of claim 17, wherein the cell culture undergoes autotrophic growth.

20. A method for producing a cannabinoid in a genetically engineered microorganism, comprising introducing into the microorganism at least one nucleic acid molecule encoding tetraketide synthase and olivetolic acid cyclase, wherein the microorganism is a microalga or a cyanobacterium.
